SEQUENCE LISTING (7 sequences)

SEQ ID NO: 1 - protein, 41 aa, synthetic construct
AVSLSKMLKKRSPLTTGVYVKMPPTEPECEKQFQPYFIPIN

SEQ ID NO: 2 - protein, 11 aa, synthetic construct
TTGVYVKMPPT

SEQ ID NO: 3 - protein, 4 aa, synthetic construct
VYVK

SEQ ID NO: 4 - protein, 4 aa, synthetic construct
YVKM

SEQ ID NO: 5 - protein, 27 aa, synthetic construct
TTGVYVKMPPTEPECEKQFQPYFIPIN

SEQ ID NO: 6 - protein, 5 aa, synthetic construct
LNGFR

SEQ ID NO: 7 - protein, 9 aa, synthetic construct
NWSHPQFEK

(19) United States
(12) Patent Application Publication
(10) Pub. No.: US 2026/0176369 A1
(43) Pub. Date: Jun. 25, 2026

(54) Activation Inducible Antigen Receptors for Adoptive Immunotherapy

(71) Applicant: Stichting Amsterdam UMC, Amsterdam (NL)

(72) Inventors: Maria Themeli, Utrecht (NL); Afroditi Katsarou, Utrecht (NL); Georgios Kladis, Amsterdam (NL)

(21) Appl. No.: 19/127,684

(22) PCT Filed: Nov. 8, 2023
(86) PCT No.: PCT/NL2023/050587

§ 371 Date: May 6, 2025

(30) Foreign Application Priority Data
Nov. 8, 2022 (EP) 22386075.0

(51) Int. Cl. C07K 16/28; A61K 40/11

(52) U.S. Cl. CPC C07K 16/2878; A61K 40/11

(57) ABSTRACT

The invention relates to an inducible chimeric co-stimulatory receptor (CCR) comprising an intracellular T cell activation dependent localization domain. The invention further relates to an immune cell expressing the inducible CCR, a nucleic acid molecule encoding said inducible CCR, and to a pharmaceutical composition, comprising said immune cell or said nucleic acid molecule. The invention further relates to a method of producing said immune cell and to a method of treating a malignancy, comprising providing immune cells expressing the inducible CCR to a patient in need thereof.

### FIELD

The invention relates to the field of therapy, specifically cancer therapy, more specifically adoptive immunotherapy for hematological malignancies and solid tumors.

### 1. INTRODUCTION

The use of genetically engineered immune cells, such as T cells, bearing tumor-antigen specific T cell receptors (TCR) or chimeric antigen receptors (CAR) holds potential for effective treatment of cancer. Many potential tumor-associated targets of T cell therapy have been tested pre-clinically and/or clinically for different types of hematological malignancies such as acute lymphoblastic leukemia (ALL), chronic lymphocytic leukemia (CLL), lymphoma, acute myeloid leukemia (AML) and Multiple Myeloma (MM). Especially, CAR-modified T cells binding CD19 for B-cell leukemias have produced impressive clinical results with 80-90% complete remissions in ALL (Davila et al., 2013. Blood 122:69; Brentjens et al., 2013. Science Transl Med 5:177).

The unprecedented success of CAR T cells in hematological malignancies resulted in a growing number of pre-clinical and/or clinical studies focused on translating this treatment to solid tumors. Current studies of CAR T cells in solid tumors such as breast tumors, brain tumors, lung tumors and genitourinary tumors, primarily evaluate potential tumor-associated targets. While therapeutic efficacy of CAR T cell therapy against hematological malignancies has been remarkable, challenges related to antigen expression heterogeneity, off-tumor/on-target toxicity, CAR T cell trafficking, expansion and persistence need to be circumvented in order to impact the entire cancer landscape including solid tumors and hematological malignancies.

The widespread application of CAR T cell therapy especially in solid tumors is limited by the lack of truly tumor-restricted targets leading to unwanted on-target/off-tumor toxicities on normal tissue. To attain broader relevance, CAR therapy must achieve effective and long lasting tumor targeting and elimination with minimal or tolerable toxicity. The paucity of tumor-restricted antigens has previously incited the development of engineering concepts to spatially control CAR T cell function. CARs of lower affinity targeting some typically solid tumor antigens such as Erbb2/HER2 or EGFR, or some typically hematological tumor antigens such as CD123 or CD38 showed better discrimination between tumors and normal tissues expressing the same antigen in lower levels (Liu X et al., 2015. Cancer Res 75:3596-607, Arcangelli et al., 2017. Mol Ther 25: 1933-1945, Drent et al., 2017. Mol Ther 25: 1946-1958). However, decreasing CAR affinity results in a higher target expression-threshold for T cell activation and, depending on the level of antigen expression on tumor cells, may also hamper the efficacy of anti-tumor function. As an alternative strategy, designs exploiting a Boolean AND-gate concept have been developed, which require the input from two antigens that are tumor-specific in combination but not necessarily individually (Kloss et al., 2013. Nat Biotechnol 31:71-75). Tumor selectivity can be secured by the use of two receptors with complementary signals such as a CAR delivering minimal T cell activation and a chimeric costimulatory receptor (CCR) for co-stimulation, provided that the two receptors target a pair of antigens co-expressed in tumor cells but not in normal cells (Kloss et al., 2013. Nat Biotechnol 31:71-75).

In 2016, Morsut et al. (Morsut et al., 2016. Cell 164:780-91; Roybal et al., 2016. Cell 167:419-432) introduced a synthetic Notch receptor (called SynNotch) transcriptional circuit to spatially control CAR expression and function utilizing combinatorial antigen recognition. The SynNotch receptor contains an antigen-binding domain targeting a first antigen and a domain containing a synthetic transcription factor (TF), namely yeast- and herpesvirus-derived Gal4-VP64 fusion protein. Both domains are linked by a transmembrane domain of the Notch-1 molecule. The TF is released upon antigen engagement through cleavage of the transmembrane domain of the Notch-1 molecule. This cleavage is mediated by the gamma-secretase complex, targeting a cleavage sites present in the transmembrane domain. The released TF moves to the nucleus, binds DNA and can promote expression of an engineered CAR encoding gene, which is under control of a GAL4 responsive promoter. The newly expressed CAR receptor protein targets a second antigen. T cell activation is mediated via binding said second antigen. SynNotch circuit activation successfully controls CAR T cell function in vitro and in vivo in various models. A humanized SynNotch receptor, termed synthetic intramembrane proteolysis receptor (SNIPR), has been described as well (Zhu et al., 2022. Cell 185:1431-1443). This system uses human-derived domains to reduce immunogenicity. A disadvantage of the SynNotch system is that it depends on transcription and translation. A further disadvantage is that T cells comprising the described SynNotch receptor are only armed and activated in the presence of dual antigen positive tumor cells, resulting in increased possibility of antigen-escape mediated relapse in case of a downregulation of the first antigen.

Besides off-tumor toxicity, CAR T cell therapy is challenged by mechanisms of disease escape. A first critical mechanism of disease escape and relapse from adoptive T cell therapy is the reduction or loss of target-antigen expression (Majzner et al., 2019. Nat Med 25: 1341-1355). CARs are highly reliant on target-antigen density and as a consequence CAR T cells lose their functionality when antigen expression drops below a threshold that depends on the type of the target and the CAR binding properties (Ramakrishna et al., 2019. Clin Cancer Res 25: 5329-5341; Majzner et al., 2020. Cancer Discov 10:702-723; Watanabe et al., 2015. J Immunol 194:911-920). A second disease escaping mechanism is the lack of long term in vivo persistence of CAR cells. Dual targeting using two CARs or by a CAR and a CCR has been shown to simultaneously enhance T cell cytotoxicity to overcome the downregulation of a target antigen and improve durability of the CAR T cells (Katsarou et al., 2021. Sci Transl Med December 8; 13 (623): eabh1962; Muliaditan T et al., 2021 Cell Rep Med December 21; 2 (12): 100457; Hirabayashi et al., 2021. Nat Cancer September; 2 (9): 904-918).

There is thus need for optimized adoptive immunotherapy strategies that have improved anti-tumor effects, when compared to the currently available adoptive immunotherapy strategies. There particularly is a need for optimized adoptive immunotherapy strategies that have limited off-tumor toxicity. Furthermore, there is a need for such strategies that are overcoming the described mechanisms of disease escape and relapse, especially the reduction or loss of the target-antigen expression and limited immune cell persistence. Especially, there is need for optimized adoptive immunotherapy strategies for solid tumors that are more effective and without off-tumor toxicities.

### 2. BRIEF DESCRIPTION OF THE INVENTION

The present invention aims to improve the efficacy of chimeric antigen receptor (CAR) immunotherapy for malignancies, including hematological malignancies and solid tumors, by employing co-expression of two engineered receptors, namely a CAR and an activation inducible chimeric costimulatory receptor. A chimeric costimulatory receptor that contains the CTLA 4 intracellular domain is expressed on the surface of the T cell only upon activation and is termed CAVI-R (CTLA-4 based Activation Inducible Receptor). The use of a combination of such inducible CCR with a CAR provided temporally and spatially controlled T cell activity, ensuring tumor specificity. The spatiotemporal expression of the inducible CCR was observed upon activation of a recombinant T cell by binding of the CAR to its target antigen in the tumor environment. The ON/OFF kinetics provided by the inducible CCR were considerably faster than other strategies described in the art that for example depend on inducible gene transcription and translation. Furthermore, the advantage of using a combination of such inducible CCR and a CAR, each targeting another antigen (i.e. dual targeting), as compared to using only a CAR, is a higher binding avidity, increased cytotoxicity and improved recognition and eradication of tumors with a very low antigen density or even after loss of one antigen. Thus relapses due to target downregulation of a target ligand can be avoided. Furthermore, the combination of a CAR and an inducible CCR facilitate cross delivery of combinatorial costimulatory signaling leading to optimal activation and expansion of the effector cell population.

The invention therefore provides an inducible chimeric co-stimulatory receptor (CCR) comprising an antigen specific binding domain; a transmembrane domain; one or more intracellular co-stimulatory signaling domain; and an intracellular T cell activation dependent domain.

Such inducible CCR is continuously expressed but becomes predominantly localized on the cell surface upon activation of said cell, without requiring transcription and/or translation. Therefore, the ON/OFF kinetics of an inducible CCR according to the invention are faster than systems depending on transcription/translation of a receptor protein prior to expression of said receptor on the cell surface, such as the SynNotch system.

The T cell activation dependent domain of an inducible CCR according to the invention, preferably comprises a phosphorylation site, preferably comprising the sequence YVKM. Most preferably, said T cell activation dependent domain of an inducible CCR according to the invention comprises or is the intracellular CTLA4 domain.

Interestingly, CARs comprising an intracellular CTLA4 domain have been described before as inhibitory CARs (Fedoroc et al., 2013. Science Translational Medicine 5: 215ra172). The CTLA4 domain in those CARs was included as a powerful inhibitory signalling domain. The CTLA4 based CARs were shown to limit T cell responsiveness despite concurrent engagement of an activating 10 receptor. Surprisingly, an inducible CCR according to the invention, traffics between cytoplasm and cell membrane similar to an endogenous CTLA4 receptor, but does not act as an inhibitor. Without being bound by theory, the fact that an inducible CCR according to the invention has a stimulating effect on the immune cell may be due to the one or more co-stimulatory domains present in the inducible CCR in between the transmembrane domain and the CTLA4 domain.

The intracellular co-stimulatory domain of an inducible CCR according to the invention preferably is an intracellular 4-1BB and/or an intracellular CD28 domain.

The transmembrane domain of an inducible CCR according to the invention preferably is a CD28 or CD8α transmembrane domain.

The antigen specific binding domain of an inducible CCR according to the invention preferably binds an antigen selected from the group consisting of the antigens listed in Table 1.

The invention furthermore relates to an immune cell, preferably a T cell or NK cell, expressing the inducible CCR according to the invention. Said immune cell preferably further comprises a chimeric antigen receptor (CAR), wherein the CAR comprises an antigen specific binding domain; a transmembrane domain; optionally one or more intracellular co-stimulatory signaling domains; and an intracellular primary signaling domain, preferably a CD3ζ intracellular primary signaling domain.

In said immune cell, preferably the antigen specific binding domain of the CAR binds a first antigen and the antigen specific binding domain of the inducible CCR binds a second antigen, wherein the first antigen differs from the second antigen, or wherein the first and second antigen are the same but the binding affinity of the CAR and the inducible CCR to said first and second antigen differ, i.e. the CAR and inducible CCR may bind to different epitopes on the same tumor target. Preferably, the first and second antigen are selected from the group consisting of the antigens listed in Table 1.

The antigen specific binding domain of the CAR preferably is a single chain binding domain, preferably a single chain Fv fragment. The antigen specific binding domain of the inducible CCR preferably is a single chain binding domain, preferably a single chain Fv fragment. In embodiments, both the antigen specific binding domain of the CAR and the antigen specific binding domain of the inducible CCR is a single chain binding domain, preferably a single chain Fv fragment.

In an embodiment, the CAR and inducible CCR both have a transmembrane CD8α domain.

The invention further provides a nucleic acid molecule that enables expression of an inducible CCR according to the invention in a suitable immune cell, preferably a human T-cell or human NK cell, preferably further enabling expression of a chimeric antigen receptor (CAR). Preferably, said nucleic acid molecule is present in a vector, preferably in a viral vector.

The invention further relates to a pharmaceutical composition comprising an immune cell according to the invention or a nucleic acid molecule according to the invention.

The invention further relates to a method of producing an non-natural immune cell according to the invention, the method comprising providing immune cells, preferably human immune cells, such as human T-cells, or human NK cells, and modifying the immune cells by enabling expression of a CAR and a inducible CCR according to the invention by the immune cells.

Furthermore, the invention relates to a method of treating a malignancy, preferably a hematological malignancy or a solid tumor, in a patient, the method comprising providing immune cells, such as T cells or NK cells, whereby said immune cells are preferably isolated from the patient; modifying the immune cells by enabling expression of a CAR and an inducible CCR according to the invention in the immune cells; and administering the modified immune cells to the patient.

Furthermore, the invention relates to a method of treating a malignancy, preferably a hematological malignancy or solid tumor, in a patient, the method comprising the administration of a pharmaceutical composition according to the invention. Preferably, said administration may be combined with surgery, radiation therapy, one or more further anti-cancer drugs or a combination thereof.

### 3. BRIEF DESCRIPTION OF THE FIGURES

FIG. 1. Design of CTLA4-based activation inducible chimeric costimulatory receptors (CAVI-R). (a) Chimeric costimulatory construct designs with the CTLA4 transmembrane domain and/or intracellular domain in different positions. TM: transmembrane, IC: intracellular. (b) Surface expression of chimeric costimulatory construct designs with the CTLA4 transmembrane domain and/or intracellular domain in different positions, after activation of cells. Expression of the tested receptors was measured with an F(ab′)₂ Fragment Goat Anti-Human antibody. (c) Design of CAVI-R constructs comprising CD28 and/or the CD8α transmembrane domain. (d) Surface expression of different receptor constructs using the CD28 and/or the 4-1BB costimulatory domain, after activation. Expression of CD38 receptors was measured with an F(ab′)₂ Fragment Goat Anti-Human antibody.

FIG. 2. ON/OFF kinetics of a CAVI-R. (a) On/off kinetics of a CAVI-R upon activation with anti-CD3/anti-CD28 magnetic beads. Expression of the CAVI-R construct was assessed by flow cytometry after 24, 48 and 72 hours. Upon removal of the beads, the decay of CAVI-R expression was measured at 16 and 40 hours after activation. Expression of CD38 CAVI-R was assessed with an F(ab′)₂ Fragment Goat Anti-Human antibody. (b) On/off kinetics of a CAVI-R upon stimulation with irradiated MM1.S cells. Double transduced T cells with B cell maturation antigen (BCMA) CAR and CD38 CAVI-R were stimulated with irradiated BCMA expressing cells and surface expression of the CAVI-R construct was measured after 24 and 48 hours. Upon removal of the antigen presenting cells, the decay of CAVI-R expression was monitored after 16 and 40 hours. Expression of CD38 CAVI-R was measured after binding of Protein L.

FIG. 3. Cytotoxic function of BCMA CAR+CD38 CAVI-R T cells. Lysis of luciferase expressing MM1.S (BCMA⁺CD38⁺) cells is shown for non-activated and activated T cells. Tumor cell killing was measured in a 16 hour bioluminescence (BLI) assay (n=4 per group) at the indicated effector:target (E:T) ratios.

FIG. 4. Ability of CAR+CAVI-R T cells to overcome loss of the CAR target antigen. (a) A schematic representation of the co-culture cytotoxicity assay is shown. mCherry-positive BCMA⁺CD38⁺ MM1.S cells were co-cultured with GFP-positive K562−CD38⁺ cells and double-transduced T cells with BCMA CAR+CD38 CCR or with BCMA CAR+CD38 CAVI-R were added in the co-culture. Lysis of MM1.S and K562 cells was determined by flow cytometry. (b) MM1.S m-Cherry cells and K562−CD38 GFP cells were co-cultured at a 1:1 E:T ratio with mock, BCMA-8.28ζ+CD38-BB, BCMA-8.28ζ+CD38-BB CTLA4 or BCMA-8.28ζ+CD38-28 CTLA4 CAR T cells and cell lysis was measured after 48 hours by flow cytometry (n=4 per group). (c) A schematic representation of the cytotoxicity assay of cells bearing only the CAVI-R target but are negative for the CAR target is shown. Double-transduced T cells with BCMA CAR+CD38 CCR or BCMA CAR+CD38 CAVI-R were firstly activated with BCMA⁺CD38⁺ MM1.S cells for 24 hours. The activated double-transduced T cells were then co-cultured with luciferase-positive K562−CD38⁺ cells and lysis was determined by BLI after 24 hours. The double-transduced T cells were then re-challenged with luciferase-positive K562−CD38⁺ cells and lysis was determined by BLI after 24 hours. To investigate if the activation induced by the CAVI-R is enough to retain the expression of the CAVI-R construct in the cell surface, the double-transduced T cells were re-challenged for a third time with luciferase-positive K562−CD38⁺ cells and lysis was determined by BLI after 24 hours. (d) MM1.S cells were co-cultured at a 1:1 E:T ratio and luciferase-positive K562−CD38⁺ cells were co-cultured at a 2:1 E:T ratio with mock, BCMA-8.28ζ, BCMA-8.28ζ+CD38-BB or BCMA-8.28ζ+CD38-BB CTLA4 CAR T cells and cell lysis was measured after 24 hours by BLI (n=4 per group). Statistical analysis was performed by a paired t-test. *p<0.05, ****p<0.0001. (e) A schematic representation of the cytotoxicity assay is shown. Double-transduced T cells with BCMA CAR+CD38 CCR or BCMA CAR+CD38 CAVI-R were firstly activated with BCMA+CD38⁺ MM1.S cells for 24 hours. The activated double-transduced T cells were then co-cultured with luciferase-positive K562−CD38⁺ cells 24, 48 and 72 hours after activation and lysis was determined by BLI. (f) Luciferase-positive K562−CD38⁺ cells were co-cultured at a 2:1 E:T ratio with mock, BCMA-8.28ζ, BCMA-8.28ζ+CD38-BB or BCMA-8.28ζ+CD38-BB CTLA4 CAR T cells and cell lysis was measured after 0, 24 and 48 hours by BLI (n=4 per group). Statistical analysis was performed by a paired t-test. ****p<0.0001.

FIG. 5. CAR+CAVI-R T cells show improved in vitro expansion. (a) CAR T cell proliferation following weekly antigen-specific stimulations with irradiated tumor cells. Black arrows indicate addition of irradiated tumor cells. The fold of the expansion of the CAR+ T cells is indicated on the y-axis. The data represent the mean±SEM of 4 experiments with different donors. Statistical analysis was performed using a two-way ANOVA and subsequent multiple comparison, corrected by Turkey test. *p<0.05, ****p<0.0001. (b) PD-1 expression is shown for T cells from the proliferation assay in (a) (n=3 donors). (c) Flow cytometry density plots of phenotypic profile of each group from (a) are shown at day 0 (before expansion) and at day 35. Cells were characterised as naive (CD45RA+/CD62L+), central memory (CD45RA−/CD62L+), effector memory (CD45RA−/CD62L−), or effector (CD45RA+/CD62L−) cells. (d) Percentage of CAR T cells from (b) that have a central memory or an effector memory phenotype at day 35 (n=3 donors).

FIG. 6. Affinity requirements of the CAR construct to induce CAVI-R expression and the impact on non-malignant hematopoietic cells. (a) Affinity characteristics are shown for anti-CD38 antibodies used to generate scFvs. The surface plasmon resonance—determined dissociation constant (KD value, in nanomolars) and half-effective concentration (EC50; in micrograms per milliliters) when titrated on Chinese hamster ovary—CD38 cells. (b) Surface expression of the CAVI-R construct after activation of the cells through low affinity CARs. Expression of the CD19 CAVI-R was measured after staining with a recombinant human CD19 Fc chimera protein antibody. (c) CD14+ monocytes from the same healthy donor were co-incubated with mock, CD38(28)−CAR+CD38−CAVI-R, CD38(B1)−CAR+CD38−CCR or CD38(B1)−CAR+CD38−CAVI-R T cells for 48 h. (d) The graph depicts lysis of the total CD14+ (monocytes) subsets at 1:1 effector to target ration, determined with flow cytometry. n=1. (e) Flow cytometry density plots of monocytes from (c) are shown.

### 4. DETAILED DESCRIPTION OF THE INVENTION

### Definitions

The singular forms “a”, “an” and “the”, as used herein, are intended to include the plural forms as well.

The term “or”, as used herein, includes any and all combinations of one or more of the associated listed items, unless the context clearly indicates otherwise (e.g. if an “either . . . or” construction is used).

The terms “comprise” and “comprising”, as used herein, are open language and specify the presence of stated features but do not preclude the presence or addition of one or more other features.

It will be understood that when a particular step of a method is referred to as subsequent to another step, it can directly follow said other step or one or more intermediate steps may be carried out before carrying out the particular step, unless specified otherwise.

The terms “malignancy”, “tumor” and “cancer”, as are used herein, all refer to a disease characterized by deregulated or uncontrolled cell growth.

The term “hematological malignancy”, as used herein, refers to a malignancy that affects blood cells and/or bone marrow cells, and includes lymphoma, leukemia, myeloma or other lymphoid malignancies such as plasmacytoma and Waldenstrom's macroglobulinemia, as well as cancers of the spleen and the lymph nodes.

The term “solid tumor”, as used herein, refers to an abnormal growth of cells that do not contain liquid or cysts. Colorectal cancer, ovarian cancer, prostate cancer, breast cancer, brain cancer, cervical cancer, bladder cancer, anal cancer, uterine cancer, liver cancer, pancreatic cancer, lung cancer, endometrial cancer, bone cancer, testicular cancer, skin cancer, melanoma, kidney cancer, stomach cancer, esophageal cancer, head and neck cancer, salivary gland cancer are non-limiting examples of different types of solid tumor cancers.

The term “adoptive immunotherapy”, also called “adoptive cell transfer” and “cellular adoptive immunotherapy”, as is used herein, refers to the administration to an individual in need thereof of immune cells, preferably T cells or NK cells, for the treatment of a disease or condition that is characterized by an insufficient or inadequate immune response. Said immune cells may be derived from the individual to be treated (autologous immune cells) or from a donor (allogenic immune cells). Adoptive immunotherapy can be an appropriate treatment for any disease or condition where the elimination of infected or transformed cells has been demonstrated to be achievable by immune cells such as T cells and NK cells. Said disease or condition may include, but is not limited to, a malignancy such as, melanoma, prostate, breast, colorectal, stomach, throat, neck, pancreatic, cervical, ovarian, bone, or lung cancer; leukemia, a viral infection such as hepatitis B, hepatitis C, human immunodeficiency virus; a bacterial infection, such as tuberculosis, leprosy and listeriosis, and a parasitic infection such as malaria. Types of adoptive cell therapy include chimeric antigen receptor T-cell (CAR T-cell) therapy.

The term “immune cell”, as used herein, refers to a cell of hematopoietic origin that plays a role in an immune response. Immune cells include lymphocytes (e.g. B cells and T cells), natural killer cells, and myeloid cells (e.g. monocytes, macrophages, eosinophils, mast cells, basophils, and granulocytes).

The term “T cell”, as used herein, refers to a type of lymphocyte that matures in the thymus. T cells play an important role in cell-mediated immunity and differ from other lymphocytes (e.g. B cells) in the presence of a T cell receptor (TCR) on the cell surface. The term T cell includes all cell types of immune cells expressing a T cell receptor (CD3), such as T helper cells (i.e. CD4+ T-cell), TH1 cells, TH2 cells and TH17 cells, and cytotoxic T cells (i.e. CD8+ T-cell). The term T cell also includes natural killer T cells, T regulatory cells (CD4+ CD25+ T cells), and γδT cells.

The term “conventional T cell”, as used herein, refers to a T cell which expresses a T cell receptor (TCR) as well as a co-receptor. Said co-receptor may be CD4 or CD8. Said co-receptor does not have an immunosuppressive function. A T regulatory cell (Treg) is not included under the term “conventional T cell”.

The term “Natural Killer (NK) cell”, as used herein, refers to a large granular lymphocyte cell involved in the innate immune response. NK cells are a subset of peripheral blood lymphocytes that are cytotoxic lymphocytes. NK cells can be defined by the expression of CD56 or CD16 and the absence of T cell receptor (CD3).

The term “T-cell receptor (TCR)”, as used herein, refers to a molecule on the surface of T cells that is responsible for recognizing fragments of antigen as peptides bound to major histocompatibility complex (MHC) molecules. A TCR is a heterodimeric protein that is often complexed with several CD3 chains and a ζ-chain (CD247) accessory molecules.

The term “chimeric antigen receptor (CAR)”, as is used herein, refers to an artificial transmembrane protein receptor minimally comprising an antigen binding domain, a transmembrane domain and an intracellular primary signaling domain, capable of activating or stimulating immune cells. The term “first generation CAR” refers to a CAR containing a single primary signaling domain. The term “second generation CAR” refers to a CAR containing a co-stimulatory signaling domain in addition to a primary signaling domain. The term “third generation CAR” refers to a CAR comprising two co-stimulatory signaling domains in addition to a primary signaling domain.

The term “chimeric co-stimulatory receptor (CCR)”, as is used herein, refers to a type of chimeric antigen receptor that mediates co-stimulation but does not provide immune cell activation. When expressed on an immune cell in combination with an antigen recognition receptor (e.g., a CAR or TCR that activates the cell), the CCR is often targeted to a second antigen, so that binding of both CAR and CCR to their respective signals provides maximal stimulation.

The term “inducible receptor” or “regulatable receptor”, as used herein, refers to a receptor, such as a CAR or CCR, for which the expression on a cell surface can be spatially and/or temporally controlled. Spatially controlled receptor expression means that the location of the expressed receptor can be controlled, e.g. expression on the cell surface versus expression in the intracellular domain. Temporally controlled receptor expression refers to the expression of a receptor which is limited in time such as a transient expression of a receptor upon activation. An example of an inducible CCR that is both temporally and spatially inducible is a CAVI-R. The localization of an inducible receptor according to the invention, is regulatable, independent of transcription or translation of the inducible receptor.

The term “T cell activation dependent domain”, as used herein, refers to a domain of an inducible CCR receptor that enables the presence of the inducible CCR on the surface of an immune cell to be dependent on the activation status of said immune cell. Said T cell activation dependent domain preferably is a T cell activation dependent localization domain. More specifically, upon immune cell activation, the immune cell expressing the inducible CCR having a T cell activation dependent domain will express said inducible CCR on the immune cell surface, while in the resting state of said immune cell the inducible CCR is not present on the surface of said immune cell. A person skilled in the art understands that when a certain receptor is expressed on the surface of a cell, the receptor is embedded in the plasma membrane of said cell via a transmembrane domain, usually with an extracellular domain that extends externally from said cell and an intracellular domain that extends into the interior of said cell. A T cell activation dependent domain preferably comprises a phosphorylation site that regulates the absence and presence (i.e. also called ON/OFF kinetics) of the inducible CCR on the cell surface. The intracellular domain of CTLA4 is an example a T cell activation dependent domain comprising a phosphorylation site. Upon activation of a T cell, either by the TCR or by the CAR, phosphorylation of the intracellular domain of CTLA4 of an inducible CCR will result in the localization of the inducible CCR on the surface of the activated immune cell. Said localization on the cell surface may be detectable within 6 hours after activation of the immune cell. When present on the cell surface, the inducible CCR may bind to its ligand. Binding to its ligand will activate the inducible CCR, resulting in co-stimulation of the CAR. Also in a case were the CAR and inducible CCR do not both have a CD8α transmembrane domain, activation of the CAR by binding to its ligand will result in full activation of the T cell to attack the opposing cell. Dephosphorylation of the intracellular domain of CTLA4 of an inducible CCR ensures that the inducible CCR will become removed from the cell surface. Expression of the inducible CCR on the cell surface decays around 16 hours after removal of the activating signal. Twenty-four hours after removal of the activating signal, no inducible CCR is detectable on the surface of the immune cell.

The term “cytotoxic T-lymphocyte-associated protein 4 (CTLA4)”, also referred to as “Cluster of differentiation 152 (CD152)”, as is used herein, refers to is a protein receptor that functions as an immune checkpoint and downregulates immune responses. CTLA4 is a member of the immunoglobulin superfamily that is expressed by activated T cells and transmits an inhibitory signal to T cells. CTLA4 is constitutively expressed in regulatory T cells but only upregulated in conventional T cells after activation. It acts as an off switch when bound to CD80 or CD86 on the surface of antigen-presenting cells. The intracellular domain of CTLA4 corresponds to the cytoplasmic part of CTLA, corresponding to amino acid residues 183-223 of NP_005205 (UniProt entry P16410). The sequence of the cytoplasmic part of CTLA is N term-AVSLSKMLKKRSPLTTGVYVKMPPTEPECEKQFQPYFIPIN-C term. The phosphorylation site of the intracellular domain of CTLA4 has been reported to reside within amino acid residues 197-207 of UniProt entry P16410 (Leung et al., 1995. J Biol Chem 270:25107-25114), which corresponds to the amino acid sequence TTGVYVKMPPT. The VYVK motif, comprising a phosphorylatable tyrosine residue, may be a minimal motif that provides the localization of CTLA4 on the cell surface after activation of a T cell (Leung et al., 1995. J Biol Chem 270:25107-25114).

The term “CTLA4-based activation inducible chimeric co-stimulatory receptor (CAVI-R)”, as is used herein, refers to a type of chimeric co-stimulatory receptor (CCR) that has controllable T cell activity. A CAVI-R typically comprises an antigen-targeting domain, followed by a transmembrane domain, one, optionally two or more, intracellular co-stimulatory signaling domains and a C-terminal CTLA4 intracellular domain. The CTLA4 intracellular domain is responsible for the inducible properties of the CAVI-R resulting in an absence of the CAVI-R on the cell surface in resting condition, while upon activation of the T cell expressing said CAVI-R, the CAVI-R becomes increasingly detectable on the cell surface. The use of a CAVI-R in combination with a CAR, targeting another antigen than the CAVI-R, ensures tumor specificity combined with a highly efficient and persistent anti-tumor function.

The term “primary signaling domain”, as used herein, refers to an intracellular domain which transduces an effector function signal and directs the cell to perform a specialized function.

The term “effector function” refers to a specialized function of a cell. The effector function of a T-cell, for example, may be cytolytic activity or helper activity including the secretion of cytokines.

The term “co-stimulatory signaling domain”, as used herein, refers to an intracellular co-stimulation domain of a CAR, a CCR, and/or inducible CCR such as CAVI-R that provides a secondary non-specific activation mechanism that stimulates the primary signaling domain of the CAR. For example, a CAR comprising a CD3ζ intracellular primary signaling domain may comprise a co-stimulatory signaling domain that stimulates the effector signal of the CD3ζ domain. Additionally, if a CAR is co-expressed together with a CCR, the effector signaling domain of the CD3ζ domain of the CAR may be stimulated by a co-stimulatory domain of the CCR. Well-known examples of co-stimulatory domains are CD28 and 4-1BB intracellular domains.

The term “Cluster of differentiation 28 (CD28)”, as is used herein, refers to T-cell-specific surface glycoprotein CD28 that provides co-stimulatory signals required for T cell activation and survival. The co-stimulatory domain is present in the cytoplasmic part of CD28 corresponding to amino acid residues 180-220 of UniProt entry P10747. The transmembrane domain is present in the transmembrane region of CD28 corresponding to amino acid residues 153-179 of UniProt entry P10747.

The term “4-1BB”, as used herein, refers to a member of the tumor necrosis factor receptor (TNFR) superfamily with an amino acid sequence provided as UniProt entry Q07011. The intracellular domain of human 4-1BB is present in the cytoplasmic part of CD137, corresponding to amino acid residues 214-255 of Uniprot entry Q07011.

The term “antigen specific binding domain”, as is used herein, refers to a domain of a TCR, a CAR, a CCR, and/or inducible CCR such as CAVI-R which specifically binds to a particular antigen. Preferably, said binding domain binds specifically to the target that is specified. While binding to other targets cannot be excluded, it is preferred that binding to another target occurs with a lower affinity, preferably at least 10× lower than the affinity of binding to the specified target. For example, when a CD38-specific binding domain of a CAR binds with a Kd of between 5 n and 10 nM to CD38, it is preferred that this CD38-specific binding domain binds to another target with a Kd of more than 50 nM to CD38 such as, for example, more than 100 nM, preferably more than 500 nM, most preferably more than 1 μM.

The term “affinity” or “binding affinity”, as used herein, refers to the apparent binding affinity, which is expressed as the equilibrium dissociation constant (Kd) between the binding domain and its epitope on a target molecule. Said binding affinity is the sum of the attractive and repulsive forces operating between the binding domain and its epitope.

The term “single-chain variable fragment (scFv)”, as is used herein, refers to a heavy chain (V_{H}) and light chain (V_{L}) of an immunoglobulin (e.g., mouse or human) that are covalently linked to form a V_{H}:V_{L} heterodimer.

The term “nucleic acid molecule”, as is used herein, refers to a molecule consisting of nucleotides which are covalently linked to each other by phosphodiester-bonds of a sugar/phosphate-backbone. Preferred nucleic acid molecules are DNA or RNA molecules. The term “nucleic acid molecule” also encompasses modified nucleic acid molecules, such as base-modified, sugar-modified or backbone-modified DNA or RNA molecules.

The term “vector”, as is used herein, refers to an isolated nucleic acid molecule which can be used to deliver a nucleic acid to the interior of a cell. Numerous vectors are known in the art including, but not limited to, linear polynucleotides, polynucleotides associated with ionic or amphiphilic compounds, plasmids, and viruses.

The term “viral vector”, as is used herein, is a vector that includes nucleic acid elements derived from viruses. Examples of viral vectors include, but are not limited to, adeno-associated viral vectors, retroviral vectors and lentiviral vectors. The term “autologous”, as is used herein, refers to any material derived from the same individual to whom it is later to be re-introduced into.

The term “allogeneic”, as is used herein, refers to any material derived from a different member of the same species as the individual to whom the material is introduced. Two or more individuals are said to be allogeneic to one another when the genes at one or more loci are not identical.

### Chimeric Receptors

An inducible CCR according to the invention comprises an antigen specific binding domain; a transmembrane domain such as a CD28 or a CD8α transmembrane domain; one, optionally two or more, intracellular co-stimulatory domain such as a 4-1BB and/or CD28 intracellular signaling domain and an intracellular T cell activation dependent domain. Said T cell activation dependent domain is responsible for the inducible properties of the inducible CCR. A preferred T cell activation dependent domain is an intracellular CTLA4 domain. Said inducible CCR comprising an intracellular CTLA4 domain is referred to herein as CTLA4-based activation inducible chimeric co-stimulatory receptor, called CAVI-R.

A CAR expressed by an immune cell according to the invention, comprises an antigen specific binding domain; a transmembrane domain, for example a CD8α or CD28 transmembrane domain; and an intracellular primary signaling domain, preferably a CD3ζ domain. In addition, a CAR may comprise one or more co-stimulatory domains, for example a 4-1BB domain and/or a CD28 intracellular domain.

The domains of the inducible CCR and/or CAR are preferably human. Receptors containing only human domains have less risk for immunogenicity.

### The T Cell Activation Dependent Domain of an Inducible CCR

An inducible CCR according to the invention is characterized by the fact the presence of said inducible CCR on the cell surface of an immune cell is dependent on the activation state of the immune cell. An inducible CCR according to the invention comprises an intracellular domain that renders the presence and absence of the inducible CCR on the surface of the immune cell to be dependent on the activation status of said immune cell. Such domain is called a T cell activation dependent domain. Preferably, such domain regulates the presence and absence of the inducible CCR on the cell surface by regulating internalization of the inducible CCR from the cell surface to the intracellular domain as well as trafficking of the inducible CCR from the intracellular domain towards the cell surface. Preferably, such T cell activation dependent domain regulates the presence and absence of the inducible CCR on the cell surface in a transcription independent manner, meaning that no new receptors need to be transcribed and/or expressed.

A preferred inducible CCR according to the invention is CAVI-R, comprising an CTLA4 intracellular domain. The presence of this domain in the CAVI-R construct is responsible for the inducible property of the CCR. Said CTLA4 intracellular domain may be fused to an intracellular co-stimulatory domain such as an intracellular CD28 or 4-1BB domain. The CTLA4 intracellular domain is preferably located at the C-terminal part of the inducible CCR.

CTLA4 is one of the most extensively studied inhibitory co-receptors. The CTLA4 receptor is characterized by unique surface expression kinetics. CTLA4 is primarily an intracellular antigen whose surface expression is tightly regulated by restricted trafficking to the cell surface and rapid internalization. Intracellular CTLA4 is found in the trans Golgi network (TGN), as well as in endosomes, secretory granules and lysosomal vesicles. Upon T-cell activation, CTLA4 is translocated to the cell surface towards the site of TCR engagement (Valk et al., 2008. Trends Immunol 29:272-279). Without being bound to theory, the mechanism involved in regulating the surface expression of CTLA4 is clathrin-dependent internalization, detailed herein below as follows. An amino acid sequence from the cytoplasmic tail of CTLA4 contains the YVKM motif which, when phosphorylated, represents a binding site for the clathrin adaptor complex AP-2. AP-2 mediates the internalisation of CTLA4 from the cell surface to endosomal and lysosomal compartments. Binding of AP-2 to CTLA4 requires non-phosphorylated Y201 in the YVKM motif. On the other hand, the phosphorylation of Y201 by Src kinases such as Lck or Fyn allows for PI3K or SHP-2 binding and prevents the interaction with AP-2, which promotes CTLA4 surface retention. Phosphorylation and dephosphorylation of Y201 thus regulate the balance between CTLA4 trafficking and signaling.

Preferably, a T cell activation dependent domain of an inducible CCR comprises a phosphorylation site that mediates the presence and absence of the inducible CCR on the cell surface. Said phosphorylation site preferably comprises the sequence YVKM, more preferably at least 5, 6, 7, 8, 9 or 10 subsequent amino acids from sequence TTGVYVKMPPT and comprising the sequence YVKM, most preferably the 11 amino acid sequence TTGVYVKMPPT.

An inducible CCR according to the invention may comprise a part of the intracellular domain of CTLA4, such as the sequence YVKM, more preferably the 11 amino acid sequence TTGVYVKMPPT, more preferably the amino acid sequence CTLA4 TTGVYVKMPPTEPECEKQFQPYFIPIN, most preferable the whole cytoplasmatic part of CTLA4.

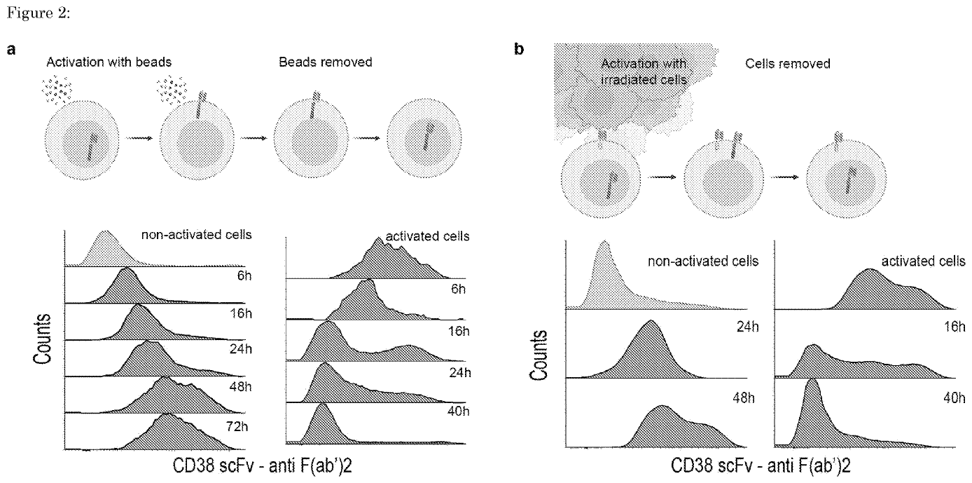

The addition of the CTLA4 intracellular domain to the inducible CCR construct was shown to result in an absence of the inducible CCR on the cell surface in resting condition, while upon activation of the immune cell expressing said inducible CCR, the inducible CCR becomes increasingly detectable on the cell surface. Based on the rapid trafficking kinetics of CTLA4-based inducible CCR (i.e. the CAVI-R), the expression on the cell surface starts within 6 hours and was shown to peak around 24-36 h post-activation of the T cell (FIG. 2). Activation of the recombinant T cells expressing both a CAR and inducible CCR can be induced by anti-CD3/CD28 antibodies, activation of a TCR or activation of a CAR. When no further activation signals are provided, the CAVI-R surface expression was shown to decay with only 30% positive cells after 16 hours and complete disappearance from the surface within 24 hours. This is considerably faster than other strategies that rely on activation inducible transcription of a gene (e.g. SNIPR, SynNotch, NFAT-promoter elements etc). When an immune cell comprises both a TCR or CAR and an inducible CCR such as a CAVI-R, the inducible CCR will only be present on the surface of the immune cell and thus be able to elicit its function when the immune cell is activated by said TCR or CAR. For example, in case of a TCR or CAR targeting a solid tumor antigen, the inducible CCR will only be present on the surface of the immune cells in the solid tumor microenvironment (i.e. the environment in which the tumor exists, including blood vessels, immune cells, fibroblasts, signaling factors, extracellular matrix, etc.) where the solid tumor antigen of the CAR or TCR is present. Similarly for a TCR or CAR targeting a hematological tumor antigen, the inducible CCR will only be present on the surface of the immune cells in the hematological tumor microenvironment (i.e. the environment in which the tumor exists, including blood vessels, immune cells, fibroblasts, signaling factors, etc.) where the hematological tumor antigen of the CAR or TCR is present.

### Intracellular Primary Signaling Domain of a CAR

An intracellular primary signaling domain of a CAR is an intracellular domain that stimulates an efficient response of the immune cell, comprising said CAR, upon binding to an antigen. The intracellular primary signaling domain of a CAR transmits an effector function signal of the cell. Preferably an intracellular primary signaling domain of a CAR comprises the intracellular portions of CD3ζ or a relevant part thereof.

CD3ζ is the zeta chain of T cell surface glycoprotein CD3. Human CD3ζ is identified by UniProt entry P20963. The intracellular domain of human CD3ζ corresponds to amino acid residues 52-164 of UniProt entry P20963. CD3ζ comprises immunoreceptor tyrosine-based activation motifs (ITAMs). Phosphorylation of these motifs results in the activation of downstream signaling pathways.

A CAR may comprise said intracellular domain of human CD3ζ, corresponding to amino acid residues 52-164 of UniProt entry P20963, or a sequence that has at least 90% identity to this domain, preferably at least 95% identity.

### Intracellular Co-Stimulatory Signaling Domains of an Inducible CCR and CAR

An intracellular co-stimulatory signaling domain of a CAR, CCR, and/or inducible CCR such as CAVI-R, provides a secondary non-specific activation mechanism that stimulates the primary signal provided by the intracellular primary signaling domain of the CAR.

Said intracellular co-stimulatory signaling domain may include the intracellular portions of CD28, 4-1BB, OX40, ICOS, CD27, CD40, 2B4, NKG2D, or a relevant part thereof, as is known to a person skilled in the art.

CD28 is the receptor for CD80 (B7.1) and CD86 (B7.2) proteins and provides co-stimulatory signals required for T cell activation and survival. The signaling domain corresponds to the cytoplasmic part of CD28, corresponding to amino acid residues 180-220 of UniProt entry P10747. This part comprises a YMNM motif, beginning at tyrosine 191, which is important for recruitment of SH2-domain containing proteins.

4-1BB, also termed CD137 or tumor necrosis factor receptor superfamily member 9, is a member of the tumor necrosis factor (TNF) receptor family. The signaling domain corresponds to the cytoplasmic part of CD137, corresponding to amino acid residues 214-255 of Uniprot entry Q07011.

OX40, also termed CD134 or tumor necrosis factor receptor superfamily member 4, is a member of the tumor necrosis factor (TNF) receptor family. The signaling domain corresponds to the cytoplasmic part of CD134, corresponding to amino acid residues 236-277 of Uniprot entry P43489.

ICOS, also termed inducible T-cell co-stimulatory or CD278, is a CD28-superfamily co-stimulatory molecule that is expressed on activated T cells. The signaling domain corresponds to the cytoplasmic part of CD278, corresponding to amino acid residues 162-199 of Uniprot entry Q9Y6W8.

2B4, also termed CD244, is a cell surface receptor expressed on natural killer cells and some T cells. The signaling domain corresponds to the cytoplasmic part of CD244, corresponding to amino acid residues 251-370 of Uniprot entry Q9BZW8.

CD27 polypeptide, also termed tumor necrosis factor receptor superfamily member 7, is a member of the TNF-receptor superfamily. This receptor plays a key role in regulating B-cell activation and immunoglobulin synthesis. The signaling domain corresponds to the cytoplasmic part of CD27, corresponding to amino acid residues 213-260 of Uniprot entry P26842.

CD40 polypeptide, also termed Tumor necrosis factor receptor superfamily member 5, is a member of the TNF-receptor superfamily. This receptor plays a key in the induction of immunoglobulin secretion in B cells. The signaling domain corresponds to the cytoplasmic part of CD40, corresponding to amino acid residues 216-277 of Uniprot entry P25942.

NKG2D is a transmembrane protein belonging to the CD94/NKG2 family of C-type lectin-like receptors. NKG2D is expressed by all NK cells, most NKT cells and subsets of γδ+ T cells (Wu et al., 1999; Jamieson et al., 2002). In addition, NKG2D is present on the cell surface of all human CD8+ T cells. NKG2D forms a complex with DNAX-activating protein of 10 kDa (DAP10) (Wu et al., 1999). The signaling domain corresponds to the cytoplasmic part of NKG2D, corresponding to amino acid residues 1-51 of Uniprot entry P26718.

A person skilled in the art will appreciate that an intracellular co-stimulatory signaling domain may have an amino acid sequence that has at least 90% identity, preferably at least 95% identity, to the indicated co-stimulatory signaling domain of CD28, 4-1BB, OX40, ICOS, CD27, CD40, 2B4, NKG2D, or part thereof.

In an embodiment, the intracellular co-stimulatory signaling domain of the inducible CCR comprises the intracellular domain of human 4-1BB and/or CD28, or a variant or variants thereof.

In an embodiment, the intracellular co-stimulatory signaling domain of a CAR comprises the intracellular domain of 4-1BB and/or CD28, or a variant or variants thereof.

### Transmembrane Domain of an Inducible CCR and CAR

An inducible CCR, such as a CAVI-R, according to the invention, comprises a transmembrane domain that is fused to an antigen specific binding domain at its N-terminus, and to a co-stimulatory signaling domain followed by a T cell activation dependent domain at its C-terminus. Said transmembrane domain may be any transmembrane domain, such as a transmembrane domain of CD4, CD8α, CD28 or CD3ζ.

CD4 is an integral membrane glycoprotein that functions as a coreceptor for MHC class II molecule: peptide complex. The transmembrane domain of CD4 corresponds to amino acid residues 397-418 of UniProt entry P01730.

The transmembrane domain of CD28 corresponds to the transmembrane region of CD28, corresponding to amino acid residues 153-179 of UniProt entry P10747.

CD3ζ, or CD247, is a nonglycosylated transmembrane protein that exists in the T-cell receptor complex as a disulfide-linked homodimer. The transmembrane domain of CD3ζ corresponds to amino acid residues 31-51 of UniProt entry P20963.

CD8α is a cell surface glycoprotein found on most cytotoxic T lymphocytes that mediates efficient cell-cell interactions within the immune system. The CD8 antigen, acting as a coreceptor, and the T-cell receptor on the T lymphocyte recognize antigen displayed by an antigen-presenting cell (APC) in the context of class I MHC molecules. The transmembrane domain of CD8α corresponds to the transmembrane region of CD8α, corresponding to amino acid residues 183-203 of UniProt entry P01732.

Again, a person skilled in the art will appreciate that an transmembrane domain may have an amino acid sequence that has at least 90% identity, preferably at least 95% identity, to the indicated co-stimulatory signaling domain of CD4, CD8α, CD28 or CD3ζ.

Depending on the selection of a transmembrane domain, the inducible CCR can provide different attributes to the immune cell comprising said inducible CCR and a CAR.

An immune cell comprising a CAR and an inducible CCR comprising an CD28 transmembrane domain, provides a localized optimal anti-tumor effect mediated by high avidity and combinatorial co-stimulation (Katsarou et al., 2021. Sci Transl Med 13: 623). The inducible CCR comprising a CD28 transmembrane domain does not mediate lysis of cells which are positive only for the inducible CCR target ligand.

An immune cell comprising a CAR and an inducible CCR comprising an CD8α transmembrane domain, wherein the CAR also comprises a CD8 α transmembrane domain, provides lysis of cells bearing only the inducible CCR target but which cells may be negative for the CAR target. This is achieved through dimerization of the CD8α transmembrane domains (Hirabayashi et al., 2021. Nat Cancer 2: 904-918). The use of an immune cell comprising a CAR and an inducible CCR both comprising a CD8α transmembrane domain, has the potential for: (1) spatially restricted targeting of antigens only within the tumor microenvironment without the risk for off-tumor toxicity; (2) dual targeting and increased cytotoxicity even at low antigen densities through increased binding avidity; (3) longer persistence through combinatorial co-stimulation; and (4) overcoming antigen-loss, since lysis can be mediated by either of the CAR and the inducible CCR.

### Antigen Specific Binding Domains

An antigen specific binding domain of an inducible CCR comprises a single chain binding domain. Preferred single chain binding domains include a single chain variable domain (scFv), a camelid VHH molecule, a shark immunoglobulin-derived variable new antigen receptor, a tandem scFv, a scFab, an improved scFab (Koerber et al., 2015. J Mol Biol 427:576-86), or an antibody mimetic such as a designed ankyrin repeat protein, a binding protein that is based on a Z domain of protein A, a binding protein that is based on a fibronectin type III domain, engineered lipocalin, and a binding protein that is based on a human Fyn SH3 domain (Skerra, 2007. Current Opinion Biotechnol 18:295-304; Škrlec et al., 2015. Trends Biotechnol 33:408-418).

An antigen specific binding domain preferably is human or humanized. De-immunization and/or humanization is often used to reduce immunogenicity of non-human molecules. De-immunization involves the identification of linear T-cell epitopes in a binding domain of interest, using bioinformatics, and their subsequent replacement by site-directed mutagenesis to non-immunogenic sequences or, preferably human sequences. Methods for de-immunization are known in the art, for example from WO098/52976.

A further preferred approach to circumvent immunogenicity of non-human binding domains that are or may be applied to humans involves humanization. Various recombinant DNA-based approaches have been established that are aimed at increasing the content of amino acid residues in binding domains that also occur at the same or a similar position in a human binding domain, while retaining specificity and affinity of the parental non-human binding domain. Most preferred amino acid residues are those residues that occur in binding domains, preferably immunoglobulin-based binding domain, that are encoded by genomic germ line sequences.

Preferred methods for humanizing antibodies are known to a person skilled in the art and include grafting of CDRs (Queen et al., 1989. PNAS 86:10029; Carter et al., 1992. PNAS 89:4285; resurfacing (Padlan et. al., 1991. Mol Immunol 28:489; superhumanization (Tan et. al., 2002. J Immunol 169:1119), human string content optimization (Lazar et al., 2007. Mol Immunol 44:1986) and humaneering (Almagro et al., 2008. Frontiers Biosci 13:1619). Further preferred methods are described in the published international applications WO2011080350; WO2014033252 and WO2009004065; and in Qu et al., 1999. Clin. Cancer Res. 5:3095-3100; Ono et al., 1999. Mol. Immunol. 36:387-395; These methods rely on analyses of the antibody structure and sequence comparison of non-human and human antibodies in order to evaluate the impact of the humanization process into immunogenicity of the final product.

Humanization may include the construction of chimeric binding bodies, preferably antibodies, in which a non-human binding domain is attached, for example by amino acid bonding, to a human protein.

The antigen specific binding domain of the CAR expressed on an immune cell according to the invention preferably is a single chain binding domain, preferably a single chain Fv fragment. The antigen specific binding domain of the inducible CCR expressed on an immune cell according to the invention preferably is a single chain binding domain, preferably a single chain Fv fragment. Most preferably, both the antigen specific binding domain of the CAR expressed on an immune cell according to the invention is a single chain binding domain, preferably a single chain Fv fragment and the antigen specific binding domain of the inducible CCR expressed on an immune cell according to the invention is a single chain binding domain, preferably a single chain Fv fragment.

If required, an antigen specific binding domain may be fused to a transmembrane domain, co-stimulatory intracellular domain and an intracellular primary signaling domain through a linking group which provides conformational flexibility so that the antigen specific binding domain can associate and bind to its epitope.

A preferred linker group is a linker polypeptide comprising from 1 to about 60 amino acid residues, preferably from 5 to about 40 amino acid residues, most preferred about 15 amino acid residues such as 10 amino acid residues, 11 amino acid residues, 12 amino acid residues, 13 amino acid residues, 14 amino acid residues, 15 amino acid residues, 16 amino acid residues, 17 amino acid residues, 18 amino acid residues, 19 amino acid residues or 20 amino acid residues.

Some preferred examples of such amino acid sequences include Gly-Ser linkers, for example of the type (Glyx Sery) z such as, for example, (Gly4 Ser)3, (Gly4 Ser)7 or (Gly3 Ser2)3, as described in WO 99/42077, and the GS30, GS15, GS9 and GS7 linkers described in, for example, WO 06/040153 and WO 06/122825, as well as hinge-like regions, such as the hinge regions of naturally occurring heavy chain antibodies or similar sequences (such as described in WO 94/04678). A most preferred linker is a (Gly4 Ser)3 linker.

A linker group may also be present between the transmembrane domain and an intracellular primary signaling domain, and/or between an intracellular primary signaling domain and a co-stimulatory domain and/or between two or more intracellular co-stimulatory signaling domains.

### Antigens

An antigen specific binding domain of a CAR and/or an inducible CCR according to the invention may target a tumor antigen that is expressed on the surface of a tumor cell, preferably a hematological tumor or a solid tumor such as a breast cancer tumor, a lung tumor, a pancreatic tumor or glioblastoma.

In case of a solid tumor, preferably said antigen specific binding domain preferably targets one or more of human epidermal growth factor receptor 2 (HER2), C-kit/cluster of differentiation 117 (CD117), Mucin 1, Mucin 16, Cancer/Testis Antigen 1, L1-cell adhesion molecule (L1CAM), Guanylyl cyclase C (GUCY2C), Folate Receptor alpha (FRα), Receptor Tyrosine Kinase Like Orphan Receptor 1 (ROR1), epithelial cell adhesion molecule (EpCAM), B7-H3, Mesothelin, Hepatocyte growth factor (HFG) receptor, GPI-anchored carcinoembryonic antigen (CEA), alkaline phosphatase placental-like 2 (ALPPL2), Intercellular Adhesion Molecule 1 (ICAM1), Chondroitin Sulfate Proteoglycan 4 (CSPG4), CD32A, Vascular Endothelial Growth Factor Receptor 2 (VEGFR2), interleukin 13 receptor subunit a2 (IL13Ra2), disialoganglioside GD2 (2R,4R,5S,6S)-2-[3-[(2S,3S,4R,6S)-6-[(2S,3R,4R,5S,6R)-5-[(2S,3R,4R,5R,6R)-3-acetamido-4,5-dihydroxy-6-(hydroxymethyl)oxan-2-yl]oxy-2-[(2R,3S,4R,5R,6R)-4,5-dihydroxy-2-(hydroxymethyl)-6-[(E)-3-hydroxy-2-(octadecanoylamino) octadec-4-enoxy]oxan-3-yl]oxy-3-hydroxy-6-(hydroxymethyl)oxan-4-yl]oxy-3-amino-6-carboxy-4-hydroxyoxan-2-yl]-2,3-dihydroxypropoxy]-5-amino-4-hydroxy-6-(1,2,3-trihydroxypropyl)oxane-2-carboxylic acid), Receptor Tyrosine Kinase AXL, Folate Hydrolase 1 (FOLH1), Prostate Stem Cell Antigen (PSCA), Prostate Specific Membrane Antigen (PSMA), Folate receptor-alpha, Glypican 3 (GPC3), CD147, epidermal growth factor receptor (EGFR) and EGFRvIII, Melanoma Antigen Gene (MAGE), Melanoma antigen recognized by T-cells 1 (MART-1), Glycoprotein 100 (gp100), CD133, Fibroblast-activation protein (FAP), Claudin 18 (CLD18), Programmed death-ligand 1 (PDL1), PRAME, Survivin and NKG2D-L.

HER2, also called Epidermal Growth Factor Receptor 2 (ERBB2), is protein tyrosine kinase that is part of several cell surface receptor complexes and is expressed in normal epidermal cells. HER2 is typically overexpressed in a breast cancer type referred to as HER2-positive breast cancer. HER2 overexpression is also reported in glioblastoma tumors.

C-kit/CD117 is a receptor tyrosine kinase. Overexpression of CD117 occurs in gastrointestinal stromal tumor, mucosal melanoma, acute myeloid leukemia, and mast cell disease (Martinez and Moon, 2019. Front Immunol 10:128).

Mucin 16 plays as role in the modulation of the composition of the protective mucus layer related to acid secretion or the presence of bacteria and noxious agents in the lumen. Is used as a tumor marker in a variety of cancers, especially ovarian cancer (Martinez and Moon, 2019. Front Immunol 10:128).

Cancer/Testis Antigen 1, also termed NY-ESO-1, is a surface antigen that is expressed in normal testis, but overexpressed in many cancers such as liposarcoma, neuroblastoma and synovial sarcoma (Martinez and Moon, 2019. Front Immunol 10:128).

L1CAM is a cell adhesion molecule, belonging to the immunoglobulin superfamily of cell adhesion molecules. L1CAM is aberrantly expressed in several different types of human solid tumors, such as ovarian cancer (Martinez and Moon, 2019. Front Immunol 10:128).

GUCY2C is a transmembrane protein that functions as a receptor for endogenous peptides guanylin and uroguanylin. Is upregulated in gastrointestinal cancers such as colorectal cancers and stomach cancers.

ROR1 is a member of the ROR receptor tyrosine kinase family and a transmembrane glycoprotein, that plays a pivotal role in cell differentiation, proliferation and survival. ROR1 is primarily expressed during embryonic and fetal development, whereas it is absent in most mature tissues. ROR1 expression was reported to elevate in human leukemia and a variety of solid malignancies such as breast cancer, lung cancer and pancreatic cancer.

EpCAM is a transmembrane glycoprotein that is expressed at the basolateral membrane of human epithelial tissues. EpCAM is reported to be overexpressed in many human epithelial cancers including colorectal, breast, gastric, prostate, ovarian, and lung cancer.

B7-H3, also known as CD276, is an immunomodulatory transmembrane N-linked glycoprotein that is overexpressed in a number of solid tumors including small cell lung cancer, non-small cell lung cancer, prostate cancer, breast cancer, glioblastoma and others.

Mesothelin is a glycosylphosphatidylinositol-anchored cell-surface protein that may function as a cell adhesion protein. This protein is overexpressed in epithelial mesotheliomas, lung cancer, pancreatic cancers, ovarian cancers and in specific squamous cell carcinomas.

EGFR is a tyrosine kinase transmembrane receptor that regulates epithelial tissue development and homeostasis. In pathological settings, mostly in lung and breast cancer and in glioblastoma, EGFR is a driver of tumorigenesis. Tumors with EGFR gene amplification frequently contain EGFR gene rearrangements, with the most common extracellular domain mutation being EGFRvIII. This mutation results in a deletion of exons 2-7 of the EGFR gene and renders the mutant receptor incapable of binding any known ligand. EGFRvIII is expressed especially in patients with glioblastoma.

IL13Rα2 is a high affinity IL13 receptor that lacks a cytoplasmic domain. It is reported to be upregulated in some cancers such as glioma including glioblastoma, breast cancer, pancreatic cancer and osteosarcoma (Martinez and Moon, 2019. Front Immunol 10:128).

GD2 is the major ganglioside present in human neuroblastoma cell lines. GD2 is synthesized in large quantities by neuroblastomas and glioblastomas. HFG receptor or c-MET, is a transmembrane protein that in humans is encoded by the MET gene. The protein possesses tyrosine kinase activity. The primary single chain precursor protein is post-translationally cleaved to produce the alpha and beta subunits, which are disulfide linked to form the mature receptor. MET is upregulated in many types of human malignancies, including cancers of kidney, liver, stomach, breast, and brain.

CEA a cell surface adhesion molecule member of the Immunoglobulin Superfamily (IgSF). Aberrant upregulation of CEA is a common feature found in a wide variety of human cancers such as colon, breast and lung cancer.

ALPPL2 is an alkaline phosphatase that can hydrolyze various phosphate compounds. ALPPL2 expression is reported in several tumor types, including breast, pancreas, lung, bladder, testicular, ovary, melanoma, uterus, renal, prostate, central nervous system, lymphoma, colorectal, mesothelioma, and leukemia.

ICAM-1 is a transmembrane molecule and a distinguished member of the Immunoglobulin superfamily of proteins that participates in many important processes, including leukocyte endothelial transmigration, cell signaling, cell-cell interaction, cell polarity and tissue stability. ICAM-1 is highly expressed in inflammatory conditions, chronic diseases and a number of malignancies such as lung cancer, gastrointestinal cancer, breast cancer and melanoma.

CSPG4 a transmembrane proteoglycan originally identified as a highly immunogenic tumor antigen on the surface of melanoma cells, is associated with melanoma tumor formation and poor prognosis in certain melanomas and several other tumor types such as breast tumors, carcinomas and sarcomas.

CD32, also known as Fc Gamma Receptor II (FcγRII or FCGR2), is a surface receptor glycoprotein belonging to the Ig gene superfamily. CD32A is one of the isoforms of CD32. CD32 can be found on the surface of a variety of immune cells. CD32 has a low-affinity for the Fc region of IgG antibodies in monomeric form, but high affinity for IgG immune complexes. CD32 has two major functions: cellular response regulation, and the uptake of immune complexes. CD32A expression has been associated with different forms of cancers such as breast cancer and colorectal cancer.

VEGFR2 is a type III receptor tyrosine kinase. This receptor acts as a cell-surface receptor for Vascular endothelial growth factor (VEGF), which is a major growth factor for endothelial cells. VEGFR2 overexpression has been reported in in neovascular tumor endothelial cells and in different types of cancer, namely breast cancer, colorectal cancer, non-small cell lung cancer, urothelial cancer, malignant melanoma and B-cell lymphoma.

AXL is a member of the TAM family with the high-affinity ligand growth arrest-specific protein 6 (GAS6). The Gas6/AXL signaling pathway is associated with tumor cell growth, metastasis, invasion, epithelial-mesenchymal transition (EMT), angiogenesis, drug resistance, immune regulation and stem cell maintenance. Aberrant expression of AXL has been shown in a number of human malignancies, including breast cancer, chronic lymphocytic leukaemia (CLL), NSCLC, pancreatic cancer, glioblastoma, melanoma, renal cell carcinoma (RCC), prostate cancer, and oesophageal cancer.

FOLH1, also termed Prostate-specific membrane antigen (PSMA), is a transmembrane protein expressed in all types of prostatic tissue. PSMA expression has been associated with prostate cancer and with other cancers such as bladder, testicular-embryonal, neuroendocrine, colon, and breast cancer, more specifically in the neovasculature associated with these cancers.

PSCA is a protein of unknown function anchored to the cell surface. It was discovered in an attempt to identify genes up regulated in human prostate cancer. Though the name implies specificity for the prostate, PSCA is expressed in several tissues such as placenta, kidney, pancreas, and bladder.

FRα is a folate-binding protein located on cellular membranes. This protein is a known cancer-associated antigen. FRα is a 38-40 kDa glycosyl-phosphatidylinositol (GPI)-anchored cell-surface glycoprotein encoded by FOLR1 and has a scarce distribution across several non-malignant tissues. FRα overexpression is reported in a number of solid tumours, such as ovarian, triple-negative breast and lung cancers.

MUC1, also called polymorphic epithelial mucin (PEM) or epithelial membrane antigen (EMA), is a mucin encoded by the MUC1 gene in humans. MUC1 is a glycoprotein with extensive O-linked glycosylation of its extracellular domain. Mucins line the apical surface of epithelial cells in the lungs, stomach, intestines, eyes and several other organs. Overexpression of MUC1 is associated with colon, breast, ovarian, lung and pancreatic cancers.

GPC3 belongs to the heparan sulfate proteoglycans family with similar structures, including a 60-70 kD core protein, which is linked to the surface of the cell membrane by a glycosylphosphatidylinositol anchor (GPI). GPC3 overexpression is associated with hepatocellular carcinoma, ovarian clear cell carcinoma, melanoma, squamous cell carcinoma of the lung, hepatoblastoma, nephroblastoma, yolk sac tumor, and some pediatric cancers.

CD147 is a transmembrane glycoprotein, also known as basigin (BSG). CD147 is a member of the immunoglobulin superfamily and plays a role in intercellular recognition. CD147 dysregulation has been found in many cancer types such as brain cancer, breast cancer, cervical cancer, lung cancer, endometrial cancer, liver cancer, colon cancer, pancreatic cancer, ovarian cancer, bladder cancer, carcinoma, melanoma, lymphoma and retinoblastoma.

The Melanoma Antigen Gene (MAGE) protein family is a large, highly conserved group of proteins that share a common MAGE homology domain. Many MAGE proteins are restricted in expression to reproductive tissues, but are aberrantly expressed in a wide-variety of cancer types including colon, melanoma, brain, lung, prostate, and breast cancer.

Melanoma antigen recognized by T-cells 1 (MART-1), also called Protein melan-A, is a transmembrane protein that is present in normal melanocytes and widely expressed in melanomas.

Glycoprotein 100 (gp 100) or Melanocyte protein PMEL is a type I transmembrane glycoprotein enriched in melanosomes and is highly expressed in melanomas.

CD133 is a transmembrane glycoprotein also known as prominin-1 that is normally expressed on undifferentiated cells including endothelial progenitor cells, hematopoietic stem cells, fetal brainstem cells, and prostate epithelial cells. CD133 is a well-known biomarker used for the isolation of cancer stem cells (CSCs). CD133 can be found in many types of cancers including gastric, breast, melanoma, lung, ovarian, pancreatic, colon, prostate, glioma and hepatocellular cancer.

Fibroblast-activation protein (FAP) is a membrane-anchored peptidase that is characteristically expressed by carcinoma-associated fibroblasts (CAFs). FAP has been reported in several cancer types including colorectal, ovarian, hepatocellular and pancreatic cancer.

Claudin 18 (CLD18) is a member of the tight junction protein family. Aberrant CLDN18 expression is associated with several cancer types including non-small-cell lung cancer, gastric cancer, breast cancer, colon cancer, liver cancer, head and neck cancer and bronchial cancer.

Programmed death-ligand 1 (PDL1) also known as cluster of differentiation 274 (CD274) or B7 homolog 1 (B7-H1) is a type 1 transmembrane protein that in humans is encoded by the CD274 gene. PDL1 has been associated with several cancer types such as lung cancer, melanoma, gastric cancer, liver cancer, urothelial cancer and lymphoma.

NKG2D is a transmembrane protein belonging to the NKG2 family of C-type lectin-like receptors. NKG2D ligands (NKG2D-L) are normally not expressed by normal tissues. NKG2D-L expression is associated with several cancer types including ovarian, cervical, breast, lung, hepatocellular, colon, renal, prostate, pancreatic and head and neck cancer, as well as in leukemia, lymphoma, multiple myeloma, melanoma, glioma, osteosarcoma and neuroblastoma.

In case of hematological tumor antigens, an antigen specific binding domain may target one or more of CD5, CD7, CD10, CD19, CD20, CD22, CD30, CD33, CD34, CD38, CD41, CD48, CD96, Purinergic Receptor P2Y, G-Protein Coupled, 10 (P2RY10), CD44v6, CD56, CD70, CD123, TNF Receptor Superfamily Member 17 (TNFRS17), Signaling Lymphocyte Activation Molecule (SLAM) Family Member 7 (SLAMF7), CD138, Kappa light chain, Lewis-Y (LeY), NY-ESO-1, POU Class 2 Homeobox Associating Factor 1 (POU2AF1), Receptor Tyrosine Kinase Like Orphan Receptor 1 (ROR1), Adhesion G Protein-Coupled Receptor E2 (ADGRE2), C-type lectin domain family 12 member (CLEC12A), C-C Motif Chemokine Receptor 1 (CCR1), Leukocyte Immunoglobulin Like Receptor B2 (LILRB2), Wilms' tumor 1 ((WT1), PRAME (PReferentially expressed Antigen in MElanoma), Survivin, HA-1 and phosphoantigens.

CD5 encodes a member of the scavenger receptor cysteine-rich superfamily, which is a type-I transmembrane glycoprotein that is present on the surface of thymocytes, T lymphocytes and a subset of B lymphocytes. CD5 is a marker for B cell chronic lymphatic leukemia, B cell small lymphocytic lymphoma, mantle cell lymphoma, malignant T cells and thymic carcinoma. CD5 may also be expressed on lymphoma's, including atypical thymoma, Burkitt lymphoma, mucosa-associated lymphoid tissue (MALT) lymphoma and splenic lymphoma.

CD7 is a cell surface glycoprotein that is present on thymocytes and mature T cells. CD7 is a marker for T-cell acute lymphoblastic leukemia and other malignant immature T cells, stem cell lymphoma, chronic myelogenous leukemia, Down syndrome associated transient myeloproliferative disorder and acute myeloid leukemia.

CD10 is a cell surface enzyme with neutral metalloendopeptidase activity. CD10 is also known as CALLA (common acute lymphocytic leukemia antigen). CD10 is a marker for the common form of acute lymphocytic leukemia as well as for Burkitt lymphoma, angioimmunoblastic T cell lymphoma, and follicular germinal center lymphoma.

CD19 is a 95 kd transmembrane glycoprotein belonging to the immunoglobulin superfamily. CD19 is classified as a type I transmembrane protein, with a single transmembrane domain, a cytoplasmic C-terminus, and extracellular N-terminus. CD19 is a marker for B cell lymphomas and leukemias.

CD20 is a membrane-embedded surface molecule which plays a role in the development and differentiation of B-cells into plasma cells. CD20 is a marker for B cell lymphomas, pre B acute lymphocytic leukemia/lymphoblastic lymphoma, spindle cell thymoma and nodular lymphocyte predominant Hodgkin lymphoma.

CD22 is a transmembrane glycoprotein member of the immunoglobulin superfamily that may bind alpha2,6-linked sialic acid-bearing ligands. CD22 is a marker for hairy cell leukemia, pre B acute lymphocytic leukemia, blastic plasmacytoid dendritic cell neoplasm.

CD30 is a cell membrane protein of the tumor necrosis factor receptor family. CD30 is a marker for anaplastic large cell lymphoma, classic Hodgkin lymphoma and primary mediastinal large B cell lymphoma.

CD33 or sialic acid binding Ig-like lectin 3 (Siglec-3) is an immunoglobulin domain comprising transmembrane receptor. CD33 is expressed on cells of myeloid and on some lymphoid cells. CD33 is a marker for Acute Myeloid Leukemia, anaplastic large cell lymphoma, chronic myeloid leukemia, chronic myelomonocytic leukemia, myeloid/granulocytic sarcoma and Burkitt's lymphoma.

CD34 is a transmembrane phosphoglycoprotein protein which may act as a cell-cell adhesion factor. CD34 is a marker for Acute Myeloid Leukemia.

CD41 is an integrin alpha chain IIb protein that in humans is encoded by the ITGA2B gene. Alpha chain 2b, together with the integrin beta 3, form a fibrinogen receptor that is expressed in platelets. CD41 is a marker for acute megakaryoblastic leukemia (AML-M7).

CD48 is a glycosylphosphatidylinositol-anchored protein (GPI-AP) found on the surface of immune cells such as NK cells, T cells, monocytes, and basophils, and participates in adhesion and activation pathways in these cells. CD48 is known to be expressed on multiple myeloma cells and other cancers of B cell origin, e.g. non-Hodgkins lymphoma, chronic lymphocytic leukemia, monoclonal gammopathy of unknown significance (MGUS), Waldenstrom's macroglobulinemia, primary/systemic amyloidosis and follicular lymphoma.

P2RY10 is a purinergic G-protein coupled receptor that is preferentially activated by adenosine and uridine nucleotides. P2RY10 has been reported to be a tumor microenvironment-associated gene and a biomarker of metastatic melanoma.

CD38 is a 46-kDa type II transmembrane glycoprotein. CD38 is uniformly highly expressed in almost all hematological malignancies, including MM, CLL, ALL, AML and lymphoma, and can thus be used as a broad hematologic malignancy-associated target.

CD44, also termed homing cell adhesion molecule (HCAM) or lymphocyte homing receptor, is a cell-surface glycoprotein involved in cell-cell interactions, cell adhesion and migration. A splice variant of CD44, termed CD44v6, is a marker for non-Hodgkin's lymphoma.

CD56, also termed neural cell adhesion molecule (NCAM), is a homophilic binding glycoprotein expressed on the surface of neurons, glia and skeletal muscle cells. CD56 is a marker for NK lymphomas.

CD70 is a cytokine that belongs to the tumor necrosis factor (TNF) ligand family and is a ligand for TNFRSF27/CD27. CD70 is expressed on the surface of activated T and B lymphocytes. CD70 is a marker for cutaneous T-cell lymphoma.

CD96, or Tactile, is a receptor protein expressed on T cells and NK cells. CD96 is a type I membrane protein belonging to the immunoglobulin superfamily. CD96 is a marker for acute myeloid leukemia.

CD123, or Interleukin (IL)-3 receptor, is a glycoprotein that, together with a Beta Common subunit, forms the heterodimeric IL3 receptor. CD123 is expressed on plasmacytoid monocytes. CD123 is a marker for leukemic stem cells.

TNFRS17, also termed B-cell maturation antigen (BCMA), is a cell surface receptor of the TNF receptor superfamily which recognizes B-cell activating factor (BAFF). BCMA is a marker for B cell leukemia, B cell lymphomas and multiple myeloma.

SLAMF7, also termed CS-1, a member of the CD2 family of cell surface receptors that is expressed on NK cells and on activated B cells. CS-1, also termed CD319, is a marker for malignant plasma cells, especially malignant myeloma plasma cells.

Kappa light chain is encoded by the immunoglobulin kappa locus on chromosome 2 and is expressed in B-cells. Kappa light chain is a marker for B-cell lymphoma and neoplastic plasma cells, such as multiple myeloma. CD138, also termed syndecan, is a transmembrane heparan sulfate proteoglycan.

CD138 is a marker for keratoacanthoma, myeloma, plasmablastic lymphoma, primary effusion lymphoma and pyothorax associated lymphoma.

LeY is an oligosaccharide (Fucα1→2Galß1→4 [Fucα1→3]GlcNAc). Although known as a blood-group epitope, LeY is overexpressed on many epithelial cancers and hematological malignancies (including AML) but has limited expression on normal healthy tissues.

NY-ESO-1, or cancer/testis antigen 1B, is a protein having no homology with any known protein. NY-ESO-1 belongs to an expanding family of immunogenic testicular antigens that are aberrantly expressed in human cancers in a lineage-nonspecific fashion. NY-ESO-1 is a marker for multiple myeloma.

POU2AF1, also termed B cell specific Octamer Binding protein-1 (BOB1), is a transcription factor that is localized intracellularly, but HLA-presenting Bob1-derived polypeptides are accessible to the cell surface of the T cell receptor (TCR) and can therefore be recognized by T cells. BOB1 is a marker for multiple hematological malignancies such as ALL, CLL, MCL and MM

ADGRE2, also termed or Egf-Like Module Containing, Mucin-Like, Hormone Receptor-Like 2 (EMR2) is a human myeloid-restricted adhesion G protein-coupled receptor. ADGRE2 is a marker for acute myeloid leukemia.

CLEC12A is a member of the C-type lectin/C-type lectin-like domain (CTL/CTLD) superfamily. Members of this family share a common protein fold and have diverse functions, such as cell adhesion, cell-cell signaling, glycoprotein turnover, and roles in inflammation and immune response. CLEC12A is a marker for acute myeloid leukemia.

CCR1, or CD191, is a member of the beta chemokine receptor family, which belongs to family of G protein-coupled receptors. CCR1 is a marker for multiple myeloma.

LILRB2, also termed CD85d, is a member of the leukocyte immunoglobulin-like receptor (LIR) family. The receptor is expressed on immune cells. LILRB2 is expressed on NK cells, T cells, monocytes/macrophages, dendritic cells and eosinophils. LILRB2 is a marker for acute myeloid leukemia.

WT1 encodes a zinc finger domain comprising DNA binding protein. WT1 is expressed at high levels in most of acute myelocytic, acute lymphocytic, and chronic myelocytic leukemia.

PRAME (PReferentially expressed Antigen in MElanoma) is a tumor-associated antigen that was first identified intumor-reactive T-cell clones derived from a patient with metastatic cutaneous melanoma.1 It was subsequently found that PRAME is not only expressed in cutaneous melanoma, but also ocular melanoma and various nonmelanocytic malignant neoplasms, including non-small cell lung cancer, breast carcinoma, renal cell carcinoma, ovarian carcinoma, leukemia, synovial sarcoma, and myxoid liposarcoma.

Survivin, also called baculoviral inhibitor of apoptosis repeat-containing 5 or BIRC5, is a member of the Inhibitor of apoptosis (IAP) family of proteins, involved in inhibition of apoptosis and regulation of cell cycle. Survivin is overexpressed in a large number of solid tumors including breast, colon, ovarian, lung, liver, uterus, glioblastoma, astrocytoma, meningioma, bladder, prostate, gastrointestinal, non-melanoma skin cancer, melanoma and soft tissue sarcoma. Survivin expression is also correlated to hematologic malignancies, including lymphoma and leukemia.

Minor histocompatibility antigen HA-1 is encoded by the polymeric HMHA1 gene and is highly expressed in leukemia cells. TCRs directed against HA-1 have been shown to be good candidates for TCR gene transfer to treat hematologic malignancies because of the hematopoiesis-restricted expression and favorable frequency of HA-1

Phosphoantigens, such as isopentenyl pyrophosphate (IPP) are overproduced in cancer cells as a result of a dysregulated mevalonate pathway (Gober et al., 2003. J Exp Med 197:163-168).

In general, an antigen specific binding domain against one of these tumor targets on either CAR or inducible CCR may be combined with an antigen specific binding domain against one further tumor target on the other chimeric receptor. For example, for breast cancer treatment, the antigen specific binding domain of the CAR may target HER2, while the antigen specific binding domain of the inducible CCR may target ROR1, EpCAM or B7-H3. Preferred breast cancer treatment options include for example a CAR targeting HER2 in combination with an inducible CCR targeting ROR1, or a CAR targeting HER2 in combination with an inducible CCR targeting EpCAM, or a CAR targeting HER2 in combination with an inducible CCR targeting B7-H3.

In case of a lung or pancreatic tumor, an antigen specific binding domain may target one or more of mesothelin, ROR1 and EGFR. More specifically, the antigen specific binding domain of the CAR may target mesothelin, while the antigen specific binding domain of the inducible CCR may target ROR1 or EFGR. Preferred lung or pancreatic cancer treatment options include for example a CAR targeting mesothelin in combination with an inducible CCR targeting ROR1, or a CAR targeting mesothelin in combination with an inducible CCR targeting EFGR.

In case of a glioblastoma, an antigen specific binding domain may target one or more of IL13Ra, GD2, EGFRVIII, B7-H3 and HER2. More specifically, the antigen specific binding domain of the CAR may target IL13Ra, while the antigen specific binding domain of the inducible CCR may target GD2, EGFRVIII, B7-H3 or HER2. Preferred glioblastoma treatment options include for example a CAR targeting IL13Ra in combination with an inducible CCR targeting GD2, or a CAR targeting IL13Ra in combination with an inducible CCR targeting EGFRvIII, or a CAR targeting IL13Ra in combination with an inducible CCR targeting B7-H3, or a CAR targeting IL13Ra in combination with an inducible CCR targeting HER2.

However, in some instances, an antigen specific binding domain on a CAR and an inducible CCR may target the same tumor target, albeit bind to different epitopes on the same tumor target. In such instance, the antigen specific binding domain of the CAR preferably has a lower affinity for its epitope on the tumor target, when compared to the affinity of the antigen specific binding domain of the inducible CCR for its epitope on the tumor target. For example, in breast cancer a CAR and inducible CCR may both bind to HER2, albeit different epitopes on HER2. In such instance, the antigen specific binding domain of a CAR has a lower affinity when compared to the antigen specific binding domain of the inducible CCR.

In addition, it was found that prior stimulation of the antigen specific binding domain of a CAR, prior to administering an immune cell comprising the CAR and inducible CCR to a patient in need thereof, may be sufficient for subsequent activation by the inducible CCR in order to efficiently target and kill tumor cells.

### Immune Cells

The invention furthermore relates to an immune cell, preferably a T cell or NK cell, expressing the inducible CCR according to the invention. Said immune cell may further comprising a chimeric antigen receptor (CAR). If said immune cell is a T cell expressing a CAR, an endogenous TCR is usually also present on the cell surface of said T cell. In some cases, such as the case of allogeneic CAR expressing T cells, the endogenous TCR may be knocked out.

Targeting two antigens instead of one may overcome antigen escape and/or antigen loss relapses. Therefore, the invention provides an immune cell, such as a T cell or NK cell, expressing a CAR and an inducible CCR such as a CAVI-R, wherein the CAR comprises an antigen specific binding domain binding to a first antigen and the inducible CCR comprises an antigen specific binding domain binding to a second antigen. In one embodiment, the first antigen differs from the second antigen. In such embodiment, it is preferred that the first and second antigen are different proteins and not different epitopes on the same protein. Alternatively, the first and second antigens are the same. In that case, preferably the CAR and the inducible CCR may bind different epitopes on the target protein. In such embodiment, the CAR may bind to the target antigen with different affinity than the inducible CCR, preferably a lower affinity.

An advantage of an immune cell expressing a CAR and an inducible CCR according to the invention, preferably whereby the CAR binds to a target antigen with a different affinity compared to the inducible CCR, preferably a lower affinity, is an improved control of activity, ensuring higher tumor specificity. As is shown in the examples, immune cells expressing an inducible CCR and a low-affinity CAR may not result in toxicity of target cells with low expression of the CAR ligand since low antigen expression is not enough to activate the T cell and induce the CCR expression. However, target cells that show high expression of the CAR ligand will induce surface expression of the inducible CAVI-R on the immune cells, resulting in specific killing of these target cells.

As is indicated herein above, in a dual targeting strategy wherein a CAR and inducible CCR target different tumor markers, each of the receptors can mediate cytotoxicity after activation of the immune cell by the CAR if both receptors comprise a CD8α transmembrane domain. This is a major improvement of the inducible receptors described in the art (e.g. SynNotch receptor). This means that even if there is downregulation of one antigen, the strategy still provides an anti-tumor effect.

TABLE 1
Antigens that may be targeted by
an inducible CCR, TCR and/or CAR.

| | Type of |
|---|---|
| Antigen | cancer |
| human epidermal growth factor | Solid cancer |
| receptor 2 (HER2) | |
| C-kit/cluster of differentiation | Solid cancer |
| 117 (CD117) | |
| Mucin 1 | Solid cancer |
| Mucin 16 | Solid cancer |
| Cancer/Testis Antigen 1, L1-cell | Solid cancer |
| adhesion molecule (L1CAM) | |
| Guanylyl cyclase C (GUCY2C) | Solid cancer |
| Folate Receptor alpha (FRα) | Solid cancer |
| Receptor Tyrosine Kinase Like Orphan | Solid cancer |
| Receptor 1 (ROR1) | |
| epithelial cell adhesion molecule (EpCAM) | Solid cancer |
| B7-H3 | Solid cancer |
| Mesothelin | Solid cancer |
| Hepatocyte growth factor (HFG) receptor | Solid cancer |
| GPI-anchored carcinoembryonic antigen (CEA) | Solid cancer |
| alkaline phosphatase placental-like 2 (ALPPL2) | Solid cancer |
| Intercellular Adhesion Molecule 1 (ICAM1) | Solid cancer |
| Chondroitin Sulfate Proteoglycan 4 (CSPG4) | Solid cancer |
| CD32A | Solid cancer |
| Vascular Endothelial Growth Factor | Solid cancer |
| Receptor 2 (VEGFR2) | |
| Interleukin 13 receptor subunit a2 (IL13Ra2) | Solid cancer |
| Disialoganglioside GD2 (2R,4R,5S,6S)-2-[3- | Solid cancer |
| [(2S,3S,4R,6S)-6-[(2S,3R,4R,5S,6R)- | |
| 5-[(2S,3R,4R,5R,6R)-3-acetamido-4,5- | |
| dihydroxy-6-(hydroxymethyl)oxan-2- | |
| yl]oxy-2-[(2R,3S,4R,5R,6R)-4,5- | |
| dihydroxy-2-(hydroxymethyl)-6-[(E)- | |
| 3-hydroxy-2-(octadecanoylamino)octadec- | |
| 4-enoxy]oxan-3-yl]oxy-3-hydroxy- | |
| 6-(hydroxymethyl)oxan-4-yl]oxy-3- | |
| amino-6-carboxy-4-hydroxyoxan-2-yl]- | |
| 2,3-dihydroxypropoxy]-5-amino-4- | |
| hydroxy-6-(1,2,3-trihydroxypropyl)oxane- | |
| 2-carboxylic acid) | |
| Receptor Tyrosine Kinase AXL | Solid cancer |
| Folate Hydrolase 1 (FOLH1) | Solid cancer |
| Prostate Stem Cell Antigen (PSCA) | Solid cancer |
| Prostate Specific Membrane Antigen (PSMA) | Solid cancer |
| Folate receptor-alpha, Glypican 3 (GPC3) | Solid cancer |
| CD147 | Solid cancer |
| epidermal growth factor receptor (EGFR) | Solid cancer |
| EGFRvIII | Solid cancer |
| Melanoma Antigen Gene (MAGE) | Solid cancer |
| Melanoma antigen recognized by T-cells | Solid cancer |
| 1 (MART-1) | |
| Glycoprotein 100 (gp100) | Solid cancer |
| CD133 | Solid cancer |
| Fibroblast-activation protein (FAP) | Solid cancer |
| Claudin 18 (CLD18) | Solid cancer |
| Programmed death-ligand 1 (PDL1) | Solid cancer |
| NKG2D-L | Solid cancer |
| CD5 | Hematological |
| | cancer |
| CD7 | Hematological |
| | cancer |
| CD10 | Hematological |
| | cancer |
| CD19 | Hematological |
| | cancer |
| CD20 | Hematological |
| | cancer |
| CD22 | Hematological |
| | cancer |
| CD30 | Hematological |
| | cancer |
| CD33 | Hematological |
| | cancer |
| CD34 | Hematological |
| | cancer |
| CD38 | Hematological |
| | cancer |
| CD41 | Hematological |
| | cancer |
| CD48 | Hematological |
| | cancer |
| CD96 | Hematological |
| | cancer |
| Purinergic Receptor P2Y, G-Protein | Hematological |
| Coupled, 10 (P2RY10) | cancer |
| CD44v6 | Hematological |
| | cancer |
| CD56 | Hematological |
| | cancer |
| CD70 | Hematological |
| | cancer |
| CD123 | Hematological |
| | cancer |
| TNF Receptor Superfamily Member | Hematological |
| 17 (TNFRS17) | cancer |
| Signaling Lymphocyte Activation Molecule | Hematological |
| (SLAM) Family Member 7 (SLAMF7) | cancer |
| CD138 | Hematological |
| | cancer |
| Kappa light chain | Hematological |
| | cancer |
| Lewis-Y (LeY) | Hematological |
| | cancer |
| NY-ESO-1 | Hematological |
| | cancer |
| POU Class 2 Homeobox Associating | Hematological |
| Factor 1 (POU2AF1) | cancer |
| Receptor Tyrosine Kinase Like Orphan | Hematological |
| Receptor 1 (ROR1) | cancer |
| Adhesion G Protein-Coupled | Hematological |
| Receptor E2 (ADGRE2) | cancer |
| C-type lectin domain family 12 | Hematological |
| member (CLEC12A) | cancer |
| C-C Motif Chemokine Receptor | Hematological |
| 1 (CCR1) | cancer |
| Leukocyte Immunoglobulin Like | Hematological |
| Receptor B2 (LILRB2) | cancer |
| Wilms' tumor 1 (WT1) | Hematological |
| | cancer |
| PRAME (PReferentially expressed | Hematological |
| Antigen in MElanoma) | cancer and |
| | solid cancer |
| Survivin | Hematological |
| | cancer and |
| | solid cancer |
| HA-1 | Hematological |
| | cancer |
| Phosphoantigens | Hematological |
| | cancer |

A further advantage of cells expressing both a CAR and an inducible CCR such as CAVI-R is that they have a higher proliferative capacity, when compared to cells without an inducible CCR. As is shown in the examples, a higher percentage of highly proliferating T cells expressing a CAR and CAVI-R retain a central memory phenotype and do not differentiate into an effector memory phenotype, when compared to cells without an inducible CCR. For example, a higher percentage of highly proliferating T cells expressing a CAR and CAVI-R show lower surface expression of programmed cell death protein 1 (PD-1), when compared to cells without an inducible CCR.

### Nucleic Acid Molecules for the Expression of Chimeric Receptors

Further provided is one or more nucleic acid molecules that enable expression of a CAR and an inducible CCR in an immune cell, preferably a human T cell or NK cell.

Said nucleic acid molecule or molecules that enable expression of a CAR and inducible CCR in an immune cell preferably is/are present in a vector. Said vector preferably additionally comprises means for high expression levels in immune cells, such as strong promoters, for example of viral origin (e.g., human cytomegalovirus) or promoters derived from genes that are highly expressed in a cell such as a mammalian cell (Running Deer and Allison, 2004. Biotechnol Prog 20:880-889; U.S. Pat. No. 5,888,809). The vectors preferably comprise selection systems such as, for example, expression of glutamine synthetase or expression of dihydrofolate reductase for amplification of the vector in a suitable recipient cell, as is known to the skilled person.

Said vector preferably is a viral vector, preferably a viral vector that is able to transduce immune cells such as T cells and NK cells. Said viral vector preferably is a recombinant adeno-associated viral vector, a herpes simplex virus-based vector, or a lentivirus-based vector such as a human immunodeficiency virus-based vector. Said viral vector most preferably is a retroviral-based vector such as a lentivirus-based vector such as a human immunodeficiency virus-based vector, or a gamma-retrovirus-based vector such as a vector based on Moloney Murine Leukemia Virus (MoMLV), Spleen-Focus Forming Virus (SFFV), Myeloproliferative Sarcoma Virus (MPSV) or on Murine Stem Cell Virus (MSCV). A preferred retroviral vector is the SFG gamma retroviral vector (Rivière et al., 1995. PNAS 92:6733-6737).

Retroviruses, including a gamma-retrovirus-based vector, can be packaged in a suitable complementing cell that provides Group Antigens polyprotein (Gag)-Polymerase (Pol) and/or Envelop (Env) proteins. Suitable packaging cells are human embryonic kidney derived 293T cells, Phoenix cells (Swift et al., 2001. Curr Protoc Immunol, Chapter 10: Unit 10 17C), PG13 cells (Loew et al., 2010. Gene Therapy 17:272-280) and Flp293A cells (Schucht et al., 2006. Mol Ther 14:285-92).

As an alternative, non-viral gene therapy may be used for generation of an immune cell expressing a CAR and inducible CCR according to the invention. Non-viral vectors include nude DNA, liposomes, polymerizers and molecular conjugates. Minicircle DNA vectors free of plasmid bacterial DNA sequences may be generated in bacteria and may express a nucleic acid acids encoding said CAR and inducible CCR at high levels in vivo.

As an alternative, an immune cell expressing a CAR and inducible CCR according to the invention may be provided by gene editing technology, including CRISPR/Cas, zinc-finger nucleases, and transcription activator-like effector nucleases-TALEN, in order to insert the receptor transgenes into specific loci with or without an exogenous promoter (Eyquem et al., 2017. Nature 543:113-117). Preferred genomic loci include the TRAC gene locus (constant region of the t cell receptor α-chain), the b2m gene locus, the AAVS1 locus and the PD-1 locus, as is known to a skilled person.

### Method of Producing

The invention further provides a method of producing an immune cell according to the invention, comprising providing immune cells, preferably human immune cells, such as human T-cells, or human NK cells, and modifying the immune cells by enabling expression of a CAR and a inducible CCR by the immune cells according to the invention. Said immune cells may be, for example, lymphocytes such as a T cells or NK cells, including primary T cells or NK cells, especially human T cells or NK cells. Methods to isolate T cells and/or NK cells from blood are known to a skilled person and include, for example, leukapheresis. It may be clear that the act of withdrawing immune cells from an individual is not necessarily part of the invention.

Isolated immune cells may be modified to enable the expression of a CAR and/or inducible CCR according to the invention. For example, an isolated immune cell may be modified to enable expression of a CAR and/or an inducible CCR in the immune cell by gene editing technology employing, for example, CRISPR-Cas or related gene editing systems. Alternatively, an isolated immune cell may be transduced or transfected with a non-viral expression cassette that enables expression of the CAR and/or inducible CCR according to the invention. Preferably, an isolated immune cell may be transduced or transfected by using a viral vector according to the invention.

A nucleic acid molecule that enables expression of a CAR and an inducible CCR may be transiently expressed in an immune cell according to the invention, such as a human T-cells or human NK cells, or may be stably integrated into the genome of the immune cell. As an alternative, said nucleic acid molecule that enables expression of a CAR and an inducible CCR may be provided on an episomally replicating vector. Examples of such episomal vectors are provided in Mulia et al., 2021. Human Gene Therapy 32:1076-1095.

Stable integration may be achieved by classical methods involving selection of cells with a selection marker that is present on the nucleic acid molecule. Such selection marker includes, for example, an antibiotic-resistant gene such as a resistance gene to zeocin, blasticidin or puromycin, or any selectable marker that can be used to select a transformed cell. Said selection preferably in performed in vitro, prior to administering the modified immune cells to a patient in need thereof. Stable integration may be targeted to one or more specific regions in the genome of an immune cell that support stable long-term expression of the nucleic acid molecules, including the TRAC gene locus, the b2m gene locus, the AAVS1 locus and the PD-1 locus. Targeting of the nucleic acid molecules to such regions may be performed using classical methods involving overlapping genomic sequences that allow homologous recombination, or gene editing technologies.

An immune cell may be stimulated with a T cell activator to stimulate proliferation and expansion of the cells. Said stimulation may be performed before or after providing the immune cells with a nucleic acid molecule that enables expression of a CAR and an inducible CCR. This stimulation may be for example performed by exposure to an activator of the T cell receptor (TCR)/CD3 complex such as Human T-Activator CD3/CD28 magnetic beads (Invitrogen), by exposure to antigen positive cells, recombinant antigens or an antibody directed to the receptor.

An immune cell comprising a nucleic acid molecule that enables the expression of a CAR and an inducible CCR may be stored upon transfection or may be immediately infused. Infusion may be performed intravenously. However, intra-tumoral, intracranial or intra-peritoneal injection are also being investigated, as well as infusion into the hepatic artery and pleural or transcatheter arterial infusion (Hartmann et al., 2017. EMBO Mol Med 9:1183-1197). To increase the tolerability of the treatment and to lower the risk of side effects, a CAR T cell dose may be split over multiple injections, such as over three injections which are each 1 day apart.

### Medicament and Methods of Treatment

The invention further provides an immune cell expressing a CAR and a inducible CCR according to the invention or a nucleic acid molecule according to the invention, for use as a medicament, preferably a medicament for treatment of a malignancy, preferably a hematological malignancy or solid tumor.

An immune cell expressing a CAR and an inducible CCR according to the invention or a nucleic acid molecule according to the invention is preferably used for prophylactic administration or therapeutic administration in humans that are suffering from a malignancy, preferably a hematological malignancy or solid tumor. Thus, an immune cell expressing a CAR and an inducible CCR according to the invention or a nucleic acid molecule according to the invention may be administered to an individual that is suspected of suffering from a malignancy, or may be administered to an individual already evidencing a malignancy in order to lessen signs and symptoms of said malignancy.

The administration of an immune cell expressing a CAR and an inducible CCR according to the invention or a nucleic acid molecule according to the invention is preferably provided in an effective amount to an individual in need thereof. An effective amount of an immune cell expressing a CAR and an inducible CCR according to the invention or a nucleic acid molecule according to the invention is a dosage large enough to produce the desired effect in which the symptoms of the malignancy are ameliorated or the likelihood of a malignancy is decreased. A therapeutically effective amount preferably does not cause adverse side effects. Generally, a therapeutically effective amount may vary with the individual's age, condition, and sex, as well as the extent of the disease and can be determined by one of skill in the art. The dosage may be adjusted by the individual physician in the event of any complication. A therapeutically effective amount may vary from about 0.01 mg/kg to about 500 mg/kg, preferably from about 0.1 mg/kg to about 200 mg/kg, most preferably from about 0.2 mg/kg to about 20 mg/kg, in one or more dose administrations daily, for one or several days. Preferred is administration of an immune cell expressing a CAR and an inducible CCR according to the invention or a nucleic acid molecule according to the invention for 2 to 5 or more consecutive days in order to effectively treat a malignancy.

An immune cell expressing a CAR and an inducible CCR according to the invention or a nucleic acid molecule according to the invention can be administered by injection or by gradual infusion over time. The administration of an immune cell expressing a CAR and an inducible CCR according to the invention or a nucleic acid molecule according to the invention preferably is parenteral such as, for example, intravenous, intraperitoneal, intranasal, or intramuscular. Preparations for parenteral administration include sterile aqueous or non-aqueous solutions suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The invention further provides a pharmaceutical composition comprising an immune cell expressing a CAR and an inducible CCR according to the invention or a nucleic acid molecule according to the invention. A pharmaceutical composition preferably comprises a pharmaceutically acceptable carrier. A carrier, as used herein, means a non-toxic material that does not interfere with the effectiveness of the biological activity of the active ingredient. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts buffers, stabilizers, solubilizers, and other materials which are well known in the art.

The invention further provides an immune cell expressing a CAR and an inducible CCR according to the invention or a nucleic acid molecule according to the invention for use in a method for treatment of a malignancy.

The invention further provides a method of treating an individual suffering from a cancer, said method comprising providing an immune cell expressing a CAR and an inducible CCR according to the invention or a nucleic acid molecule according to the invention to an individual in need thereof to thereby treat the individual.

The invention further provides a method of treating a malignancy, preferably a haematological malignancy or a solid tumor, in a patient by administrating the pharmaceutical composition according to the invention. Said method may be combined with surgery, radiation therapy, one or more further anti-cancer drugs or a combination thereof.

Said one or more further anti-cancer drugs may include chemotherapeutic drugs such as an alkylating agent, for example a nitrogen mustard such as bendamustine, chlorambucil, cyclophosphamide, ifosfamide, mechlorethamine, and melphalan, a nitrosourea such as carmustine, lomustine, and streptozocin, an alkyl sulfonate such as busulfan, a triazine such as dacarbazine and temozolomide, and an ethylenimine such as altretamine and thiotepa; an antimetabolite such as 5-fluorouracil, hydroxyurea and methotrexate; an alkaloid such as taxane and camptothecan; a mitotic inhibitor such as vinblastine, paclitaxel and etoposide; an antitumor antibiotic such as anthracycline and chromomycin; an topoisomerase inhibitor such as camptothecin.

Said one or more further anti-cancer drugs may also include one or more molecules for targeted therapy, immunotherapy and/or hormone therapy.

A molecule for targeted therapy is a molecule that specifically blocks growth of cancer cells by interfering with specific targeted molecules which are necessary for carcinogenesis and tumor growth, such as a tyrosine-kinase inhibitor and a phosphoinositide 3-kinase inhibitor, and/or a molecule that overcome inhibition of the immune system of the individual to kill cancer-associated tumor cells. Examples of the latter are immune checkpoint inhibitors such as anti-PD-1, anti-PD-L1 and anti-CTLA4, CTLA B7-1 and CTLA B7-2 molecules.

A molecule for immunotherapy is either a molecule that directs the immune system to attack tumor cells directly by targeting antigens displayed on tumor cells, and/or a molecule such as an antibody that targets antigens displayed on tumor cells.

A molecule for hormone therapy is a molecule that blocks and/or lowers a concentration of one or more specific hormones. This can be performed by either blocking the ability of an individual to produce said specific hormone or said specific hormones, or by interfering with how specific hormones behave in the human body. Some cancers, such as breast, prostate, ovarian and endometrial cancer require hormone stimulation such as steroid stimulation, to grow and/or develop. Hormone therapy specifically prevents the growing and division of hormone dependent/sensitive cancer cells. Examples of such hormone therapeutic molecules are hormone antagonists such as flutamide, goserelin, mitotane and tamoxifen, and aromatase inhibitors such as anastrozole, exemestane and letrozole.

The term “combination”, as is used herein, refers to the administration of a CAR-T cell as defined herein, with one or more further anti-cancer drugs, to an individual in need thereof. Said cyclic peptide and one or more further anti-cancer drugs may be provided in one pharmaceutical preparation, or as two or more distinct pharmaceutical preparations. When administered as two distinct pharmaceutical preparations, they may be administered on the same day or on different days to a patient in need thereof, and using a similar or dissimilar administration protocol, e.g. daily, twice daily, biweekly, orally and/or by infusion. Said combination is preferably administered repeatedly according to a protocol that depends on the patient to be treated (age, weight, treatment history, etc.), which can be determined by a skilled physician.

The invention further provides use of an immune cell expressing a CAR and an inducible CCR according to the invention or a nucleic acid molecule according to the invention in the preparation of a medicament for treating an individual suffering from a malignancy or suspected to suffer from a malignancy.

A preferred method of treating a malignancy, preferably a hematological malignancy or a solid tumor, comprises isolating immune cells, preferably T cells or NK cells, from the patient; modifying the immune cells by enabling expression of a CAR and an inducible CCR according to the invention, for example by introducing a vector according to the invention in said isolated immune cells; and administering the modified immune cells to the patient.

The immune cell expressing a CAR and an inducible CCR according to the invention or the nucleic acid molecule according to the invention will attach to cancer cells, as is indicated herein above, and couple to T-cells and/or NK cells to thereby elucidate an immune response against the cancer cells that will reduce or even eliminate said cells. The small size of an immune cell expressing a CAR and an inducible CCR according to the invention or a nucleic acid molecule according to the inventio, render said immune cell expressing a CAR and an inducible CCR according to the invention or said nucleic acid molecule according to the inventio as an ideal treatment tool for cancer. These immune cells expressing a CAR and an inducible CCR according to the invention or nucleic acid molecules according to the invention provide great promise for treatment of cancer.

### 5. EXAMPLES

### Example 1: Performance of an ActiVation Inducible Chimeric Costimulatory Receptor (CAVI-R)

### Materials and Methods

### Cell Lines

Human cell lines MM1.S, K562 (American Type Culture Collection, ATCC), U266 (ATCC) were cultured in RPMI-1640 (Thermo Fisher Scientific) with 10% HyClone Fetal Clone I (Thermo Fisher Scientific) and antibiotics (penicillin; 100 U/mL, streptomycin; 100 mg/mL). K562 cells were lentivirally transduced to express human CD38 and sorted for gradually increasing concentrations of BCMA expression. All cell lines were checked with Short Tandem Repeat (STR) analysis and were regularly tested for mycoplasma.

Primary Cells from MM Patients and Healthy Individuals.

Healthy donor peripheral blood mononuclear cells (PBMCs) from buffy coats (Sanquin blood-bank) were isolated by Ficoll-Paque (GE Healthcare Life Sciences) density centrifugation. Isolated cells were cryopreserved in liquid nitrogen until use. All primary samples were obtained after informed consent and approval by the institutional medical ethical committee.

### CAR and CCR and CAVI-R Vector Constructs

CAR, CCR and CAVI-R constructs were cloned into SFG γ-retroviral vectors using standard molecular biology techniques. BCMA-CAR constructs, employing an extracellular single chain variable fragment (scFv) have been described in WO2016/094304 A2 (BCMA02, drug product name bb2121). Four different BCMA-CAR constructs were used in the experiments: (1) construct “BCMA-ζ” was composed of a BCMA scFv, followed by a CD8α transmembrane domain and a CD3ζ signaling domain; (2) construct “BCMA-8.BBζ” was composed of a BCMA scFv, followed by by a CD8α transmembrane domain and 4-1BB and CD3ζ signaling domains; (3) construct “BCMA-28.28ζ” was composed of a BCMA scFv, followed by a CD28 transmembrane and intracellular sequence fused to CD3ζ intracellular domain; and (4) construct “BCMA-8.28ζ” was composed of a BCMA scFv, followed by a CD8α transmembrane, a CD28 intracellular sequence and a CD3ζ intracellular domain. The CAR sequences were linked by a P2A self-cleavable peptide sequence (Wang et al., 2015. Sci Rep 5: 16273) to a dsRed fluorescent expression marker or to a truncated human low-affinity nerve growth factor receptor (LNGFR) sequence. For the CCR construct the CD38-specific scFvs, previously described (Drent et al., 2017. Mol Therapy 25: 1946-1958), were followed by a CD28 transmembrane and intracellular sequence and the 4-1BB intracellular domain or by a CD8α transmembrane domain and the 4-1BB signaling domain. The CCR sequences were linked by a P2A element to a truncated LNGFR sequence. Different CAVI-R construct were used in the experiments: (1) construct “CD38-8.BB CTLA4” was composed of a CD38-specific scFvs followed by a CD8α TM domain, a 4-1BB IC and a CTLA4 IC domain at C-terminal part; (2) construct “CD38-CTLA4 BB” was composed of a CD38-specific scFvs followed by CTLA4 TM domain, CTLA4 IC domain and a 4-1 BB IC domain; (3) construct “CD38-28.28 CTLA4” was composed of a CD38-specific scFvs followed by a CD28 TM domain, a CD28 IC domain and a CTLA4 IC domain; (4) construct “CD38-28.BB CTLA4” was composed of a CD38-specific scFvs followed by a CD28 TM domain, a 4-1BB IC domain and a CTLA4 IC domain; (5) construct “CD38-28.28BB CTLA4” was composed of a CD38-specific scFvs followed by a CD28 TM domain, a CD28 IC domain, a 4-1BB IC domain and the CTLA4 IC domain; (6) construct “CD38-8.28 CTLA4” was composed of a CD38-specific scFvs followed by a CD8 TM domain, a CD28 IC domain and a CTLA4 IC domain; (7) construct “CD38-8.28BB CTLA4” was composed of a CD38-specific scFvs followed by a CD8α TM domain, a CD28 IC domain, a 4-1BB IC domain and a CTLA4 IC domain. The CAVI-R sequences were linked by a P2A element to a truncated LNGFR sequence. A strep tag II coding for the amino acid sequence NWSHPQFEK (flanked by two G4S linkers on both sides) was cloned into the CAVI-R constructs between the scFv and CD28 transmembrane domain, to detect surface expression of CAVI-R (CD38-SII-28.28 CTLA4).

### Generation of Retroviral Particles and Transduction of T Cells

Phoenix-Ampho packaging cells were calcium phosphate transfected with 10 μg CAR-expressing constructs. 16 hours post-transfection, complete medium (DMEM with 10% FBS) was refreshed. Two and three days after transfection, cell free supernatants containing retroviral particles were collected and directly used for transduction. Peripheral blood mononuclear cells (PBMCs) from healthy donors (3×10E6 per well) were stimulated with lectin-like phytohemagglutinin (PHA-L) (Roche) in a 6-well plate (Greiner Bio-One) in culture medium (RPMI-1640 with 10% FBS, 100 U/ml penicillin, and 100 μg/ml streptomycin). After 48 hours, 3×10E6 cells per ml were transferred to retronectin coated (15 μg/ml) (Takara) 6-well plates (Falcon). Retroviral transduction was performed by addition of 2 ml virus per well followed by spinoculation (1500 g for 1 hour at room temperature) in the presence of 4 μg/ml Polybrene. A second transduction was conducted after 16 hours, replacing two-thirds of the cell supernatant with freshly obtained virus (2 ml). Six to eight hours after the second transduction, half of the cell supernatant was replaced by fresh RPMI-1640 with 10% FBS and 50 IU/ml recombinant human (rh) IL-2 (Proleukin, Novartis). Transduction efficiencies were determined 72 hours later by flow cytometric detection of LNGFR (CD271) or dsRed expression. In order to isolate double-transduced CAR+CCR and CAR+CAVI-R T cells, an EasySep allophycocyanin (APC) Positive Selection Kit II (Stemcell Technologies) was used as per manufacturer's instructions to isolate T cells labeled with an CD271 (NGFR)-APC antibody (CD271; clone ME20.4, BioLegend) (staining with 3 μg per mL of sample, incubation for 15 minutes at room temperature), by positive selection. Sorted T cells bearing the CD38 CCR or CD38 CAVI-R (a mix of double CAR+CCR transduced and CCR-transduced) were further used in assays, assuming that singular CD38 CCR and CD38 CAVI-R transduced T cells would remain non-functional upon antigen engagement.

Activation with Beads or Irradiated Cells

CAVI-R T cells were activated with anti-CD3/anti-CD28 magnetic beads, DYNABEADS™ (Catalog number: 40203D from Thermo Fisher Scientific Inc.).

CAVI-R T cells were activated with irradiated U266 cells (BCMA⁺CD38⁻) or MM1.S cells (BCMA⁺CD38⁺) (60 Gy) in a 1:1 ratio, in a 6-well plate (Greiner Bio-One) in culture medium (RPMI-1640 with 10% FBS, 100 U/ml penicillin, and 100 μg/ml streptomycin).

### Bioluminescent Imaging Based Cytotoxicity Assay

Seven to ten days after transduction, serial dilutions (effector:target 2:1, 1:1, 1:2) of CAR T cells were incubated with luciferase-expressing cell lines. The luciferase signal produced by surviving cells was determined after 16 to 24 hours with a GloMax 96 Microplate Luminometer (Promega) within 15 minutes after the addition of 125 μg/mL beetle luciferin (Promega). Percent lysis was calculated as: % lysis=1−(BLI signal in treated wells/BLI signal in untreated wells)×100%.

### Monocyte Isolation

To isolate CD14+ monocytes from PBMCs, an EasySep FITC Positive Selection Kit II (STEMCELL Technologies) was used per the manufacturer's instructions to isolate T cells labeled with an CD14-FITC antibody (clone HCD14, BioLegend) by positive selection. Cells were stained with 3 g/ml of sample and incubated for 30 min at 4° C.), Sorted monocytes cells were further used in a cytotoxic assay.

### Monocyte Cytotoxic Assay

Isolated monocytes (10,000 cells) were plated in a 96-well flat-bottom plate. 10,000 CAR T cells were added in each well and were incubated for 48 hours. After incubation, the wells were washed with PBS twice and detached from the wells with Trypsin/EDTA solution (Lonza) for 20 min. Monocytes were stained with CD14 (clone HCD14, BioLegend) and Zombie-Aqua Fixable Viability Kit (Biolegend). Staining was performed with 1 μg/ml of sample, incubation for 30 min at 4° C. in PBS with 1% BSA, 10% FBS, and 0.1% sodium azide). After addition of Flow-Count Fluorospheres (Beckman Beckman 7547053), viable cells were quantitatively analyzed through Flow-Count-equalized measurements. Flow cytometry was performed on BD LSRFortessa.

### Proliferation Assay

CAR, CAR+CCR and CAR+CAVi-R T cells were counted and stimulated weekly with irradiated (60 Gy) BCMA⁺CD38⁺ MM1.S cells. Starting 7 days after transduction, 0.5×106 CAR T cells were seeded in a 24-well plate containing 0.25×106 MM1.S, to final volume of 1 mL. No additional cytokines were added.

### Flow Cytometry

Flow cytometry was performed on BD LSR Fortessa. Transduction efficiency was measured with an APC-conjugated antibody toward NGFR (CD271; clone ME20.4, BioLegend) and Protein L for CCR, whereas for CARs it was measured in the phycoerythrin (PE) channel to detect dsRed and AffiniPure F(ab′)₂ Fragment Goat Anti-Mouse IgG (Jackson ImmunoResearch). The following antibodies were used for flow cytometry staining: recombinant human CD19 Fc chimera protein (Biotechne), Anti-human CD366 (TIM-3) (clone F38-2E2, BioLegend or clone F38-2E2, Thermo Fisher Scientific), Anti-human CD279 (PD-1) (clone EH12.2H7, BioLegend or clone eBioJ105, Thermo Fisher Scientific), Anti-human CD62L (clone DREG-56, BioLegend), CD45RA (clone HI100, Thermo Fisher Scientific), LAG-3 (CD223; clone 3DS223H, Thermo Fisher Scientific) (staining 1 μg per mL of sample, incubation for 30 minutes at 4° C. in PBS with 1% BSA, 10% FBS and 0.1% NaN3 sodium azide). Flow cytometry data analysis was performed with FCS Express 6 Flow Cytometry software (De Novo Software).

### Statistical Analysis

Data analysis and visualization was performed using GraphPad Prism 8.2.1 (GraphPad Prism, RRID:SCR_002798) software. No pre-specified effect size was used to determine sample sizes. Graphs represent individual values=standard error of the mean (SEM). Normality of the data was confirmed with the Kolmogorov-Smirnov test. The statistical tests that were used to calculate the p values are described in the relevant figure legends. Differences were considered significant at p<0.05 and p values are denoted with asterisks as follows: p>0.5, not significant (ns), *p<0.05, **p<0.01, ***p<0.001, ****p<0.0001.

### Results

To test the concept of an activation inducible chimeric receptor, constructs were generated that contain a CTLA4 transmembrane domain (TM) and/or CTLA4 intracellular (IC) domain, and one or two costimulatory domains, i.e. 4-1 BB and/or CD28.

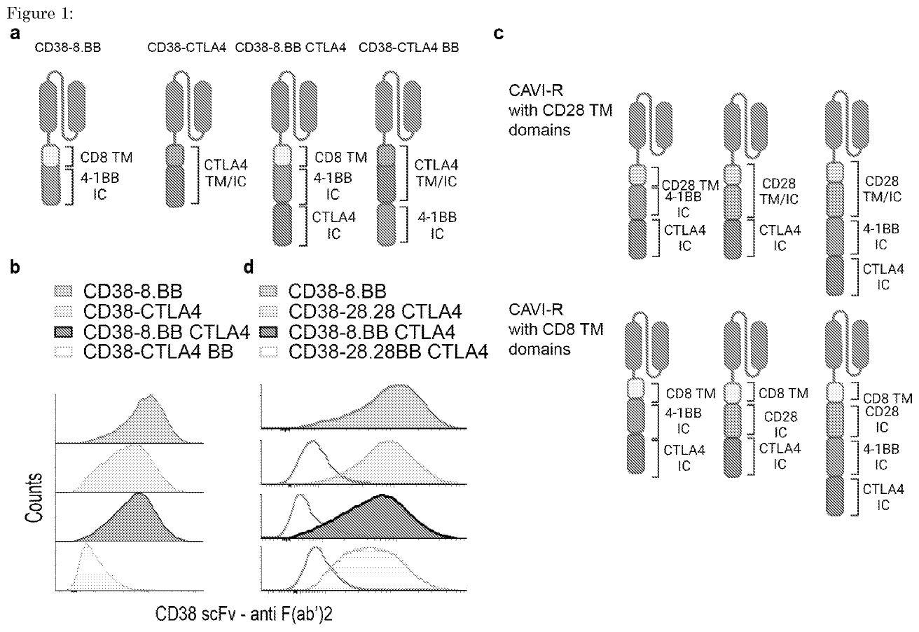

As a first model, an scFv specific for CD38 was used. CD38 is highly expressed in almost all hematologic malignancies. The CD38 CTLA4-based ActiVation Inducible chimeric costimulatory Receptor (CAVI-R) construct was designed either to contain only the CTLA4 IC domain at terminal part of the costimulatory receptor (i.e. design “CD38-8.BB CTLA4”), or to contain the CTLA4 TM and CTLA4 IC domains followed by a costimulatory domain (i.e. design “CD38-CTLA4 BB”) (FIG. 1a).

Peripheral blood T cells were transduced with the CD38.8-BB CTLA4 and the CD38-CTLA4 BB constructs and were activated with Dynabeads™ Human T-Activator CD3/CD28 for 24 hours. Upon activation, the CD38-8.BB CTLA4 receptor became increasingly detectable on the T cell surface, while CD38-CTLA4 BB T cells showed absence of expression of the receptor defining that the optimal position of the intracellular CTLA4 domain in order to achieve inducibility is at the distal to the membrane part of the CCR construct (FIG. 1b).

Further CAVI-R constructs were designed either to contain one of the well-known costimulatory domains CD28 and 4-1BB (CD38-28.28 CTLA4 or CD38-8.BB CTLA4), or containing them both (CD38-28.28BB CTLA4) (FIG. 1c). The combination of any costimulatory domain or the combination thereof (CD28, 4-1BB or 28BB) together with the CTLA4 IC domain in the CAVI-R construct resulted in absence of expression at resting condition, while upon activation receptors became increasingly detectable on the cell surface (FIG. 1d).

Further, the dynamics of the activation-induced CAVI-R expression were characterized, upon activation either with beads or through a CAR (FIGS. 2a and b). Single CAVI-R transduced T cells (CD38-8.BB CTLA4) were activated with Dynabeads™ Human T-Activator CD3/CD28. Expression of the CAVI-R was detectable 6 hours after activation, while the expression peaked around 24-36 hours post-activation (FIG. 2a). After 24 hours of stimulation, the beads were removed and the decay of the CAVI-R expression was monitored over 40 hours. CAVI-R expression level was reduced at 6 hours after bead removal. Sixteen hours after, the majority of the cells were not expressing the CAVI-R at all, while at 40 hours after removal of the beads all the cells were negative (FIG. 2a). Moreover, the dynamics of T cell activation were analyzed when stimulated through a CAR (FIG. 2b). Peripheral blood T cells transduced with BCMA-ζ CAR+CD38-8.BB CTLA4 combination were exposed to irradiated U266 cells (BCMA+CD38). The BCMA CAR+CD38 CAVI-R T cells induced CAVI-R expression in response to tumor cell stimulation within the first 24 hours, while peak expression was reached between 24-48 hours (FIG. 2b). After 24 hours of stimulation with irradiated MM cells, the MM cells were removed and the CAVI-R expression was monitored over 40 hours. The CAVI-R expression decayed with only 30% positivity after 16 hours and complete disappearance within 40 hours.

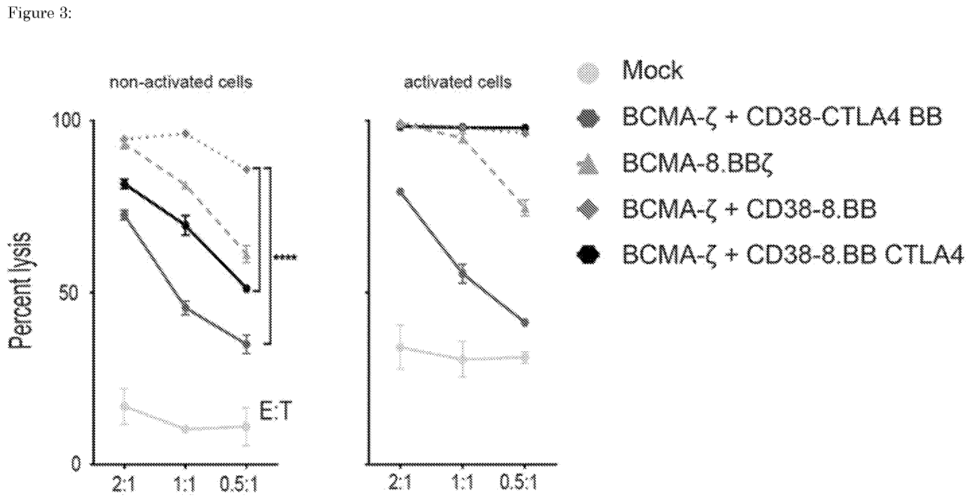

The addition of CD38−CAVI-R in first-generation BCMA-CAR T cells (BCMA-ζ) resulted in remarkable increase in their cytotoxic capacity against MM cell lines, after activation of the cells through the CAR. Second generation BCMA-CAR T cells (BCMA-8.BBζ) and double transduced BCMA-ζ+CD38-8.BB, BCMA-ζ+CD38-8.BB CTLA4 and BCMA-ζ+CD38-CTLA4 BB T cells were co-cultured with MM1.S cells (BCMA+CD38+) and the induced lysis was measured 24 hours later. The experiment was performed with cells at resting condition and upon activation. Overall, lysis of MM tumor cells by BCMA-ζ+CD38-8.BB CTLA4 was significantly higher after activation of the cells, as compared to lysis by the cells on resting condition (FIG. 3). Furthermore, BCMA-ζ+CD38-CTLA4 BB T cells performed similarly before and after activation (FIG. 3). These findings indicate that expression of an inducible CD38−CAVI-R by a CAR T cell result in a similar cytotoxic potential compared to a CD38−CCR permanently expressed on a CAR T cell. A major advantage of the inducible CD38−CAVI-R is that it only results in a cytotoxic effect similar to a permanently expressed CD38−CCR upon activation.

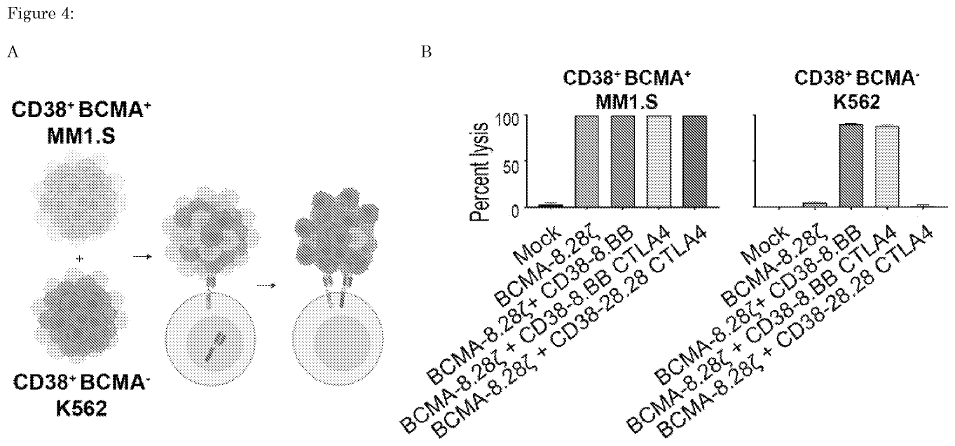

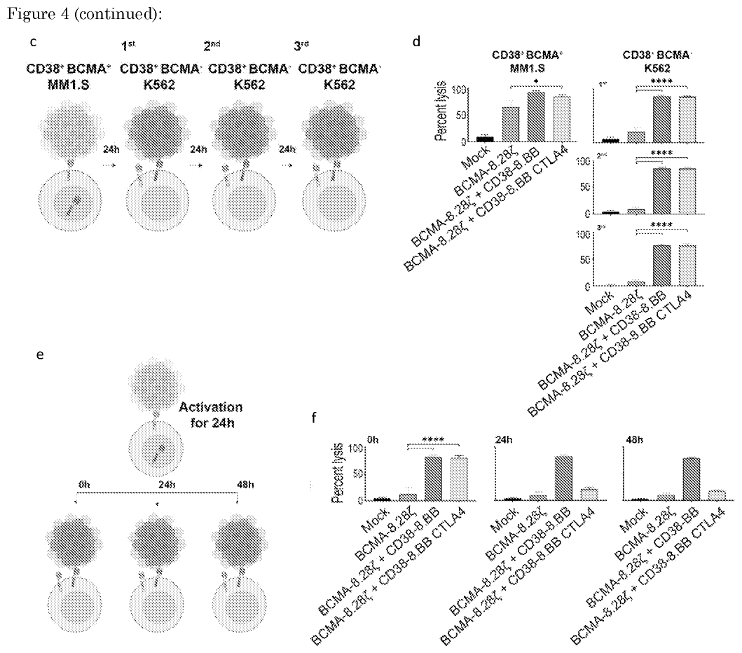

Given the superior anti-tumor lytic function against double positive MM cells (BCMA+CD38+) of the CAR+CAVI-R T cells as compared to single CAR-transduced T cells (FIG. 3), it was further tested whether the CAR+CAVI-R T cells would be able to overcome loss of the CAR target antigen. To simulate this, double positive MM1.S cells (BCMA⁺CD38⁺) were co-cultured with single positive K562−CD38+ cells (BCMA⁻CD38⁺), mimicking an antigen-escape-mediated relapse (FIG. 4a). When activated BCMA-CAR (BCMA-8.28ζ), BCMA-CAR+CD38−CCR (BCMA-8.28ζ+CD38-8.BB) and BCMA-CAR+CD38−CAVI-Rs (BCMA-8.28ζ+CD38-8.BB CTLA4 and BCMA-ζ+CD38-28.28 CTLA4) T cells were added in the coculture, the permanent expressed CCR and the inducible CAVI-R T cells with the CD8α TM domain could mediate T cell lytic function even when the CAR antigen was absent (FIG. 4b). The BCMA-8.28ζ and the BCMA-8.28+CD38-28.28 CTLA4 T cells were able to eradicate the MM1.S cells, but left the K562−CD38⁺ cells intact (FIG. 4b).

It was further tested if the activation signal induced through the CAVI-R mediated killing would be sufficient to retain the CAVI-R construct in the cell surface. To this end, the BCMA-CAR (BCMA-8.28ζ), the BCMA-CAR+CD38−CCR (BCMA-8.28ζ+CD38-8.BB) and the BCMA-CAR+CD38−CAVI-R (BCMA-8.28ζ+CD38-8.BB CTLA4) T cells were cocultured with double positive MM1.S cells (BCMA⁺CD38⁺) (FIG. 4 c). Twenty four hours later, the cytotoxicity was measured and the T cells were challenged with K562−CD38⁺ cells (BCMA⁻CD38⁺) (FIG. 4c). Twenty four hours later, killing against the CD38 single positive cells was evaluated and the T cells were re-challenged with K562−CD38⁺ cells (FIG. 4c). Killing was measured after 24 hours and K562−CD38⁺ cells were added in the culture. BCMA-CAR+CD38−CAVI-R (BCMA-8.28ζ+CD38-8.BB CTLA4) T cells preserved their cytotoxicity against K562−CD38⁺ cells, after two rounds of stimulation with CD38 single positive cells and 72 hours after the last stimulation with cells expressing the CAR target antigen (FIG. 4d). In the 3rd measurement, the inducible CD38−CAVI-R transduced cells performed similarly with the permanent CD38−CCR transduced cells, indicating that the killing induced thought the CD38−CAVI-R provides enough activation to the T cell to retain the CD38-CAVI-R expression on the cell surface.

Given the fast on/off kinetics of the CAVI-R construct, it was examined if the BCMA-CAR+CD38−CAVI-R could induce cell lysis 24 and 48 hours after activation of the T cells. Single transduced BCMA-CAR T cells (BCMA-8.28ζ), double-transduced T cells with BCMA-CAR+CD38−CCR (BCMA-8.28ζ+CD38-8.BB) and BCMA CAR+CD38 CAVI-R (BCMA-8.28ζ+CD38-8.BB CTLA4) T cells were activated with BCMA⁺CD38⁺ MM1.S cells for 24 hours (FIG. 4e). The activated double-transduced T cells were then co-cultured with K562−CD38⁺ cells 0, 24 or 48 hours later (FIG. 4e). The BCMA-8.28ζ+CD38-8.BB CTLA4 T cells preserved their cytotoxicity against the K562−CD38⁺ cells after activation, while leaving the CD38 positive cells intact when challenged 24 and 48 hours after activation (FIG. 4f). Based on these data, the decay of induced CAVI-R expression was found to be 24 hours after activation, which is likely faster than the time required for migration out of the tumor.

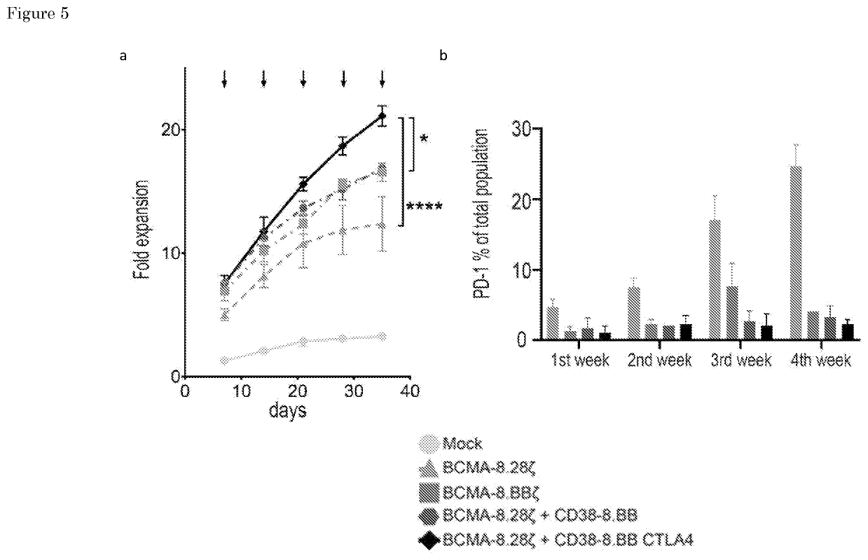

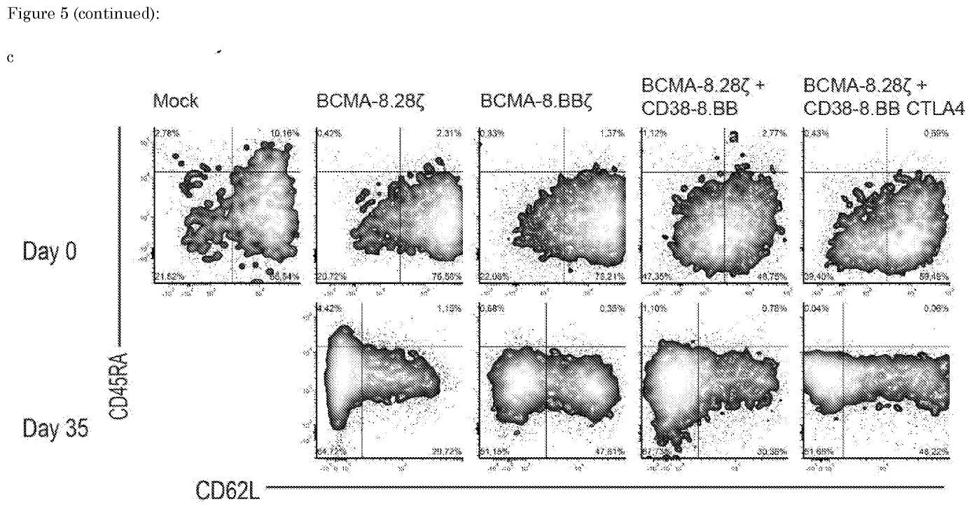

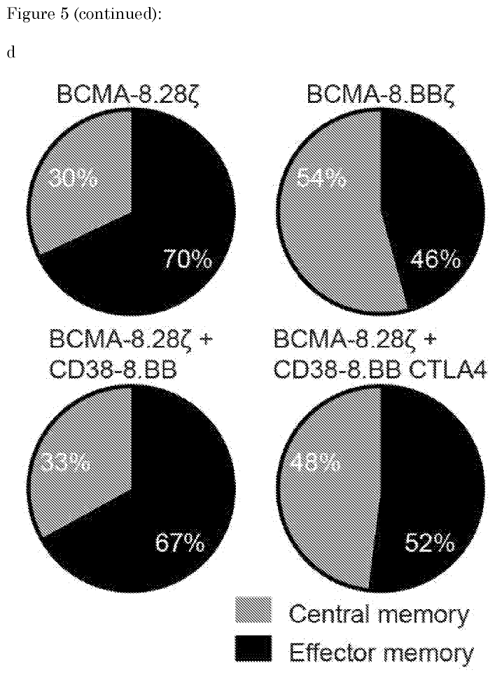

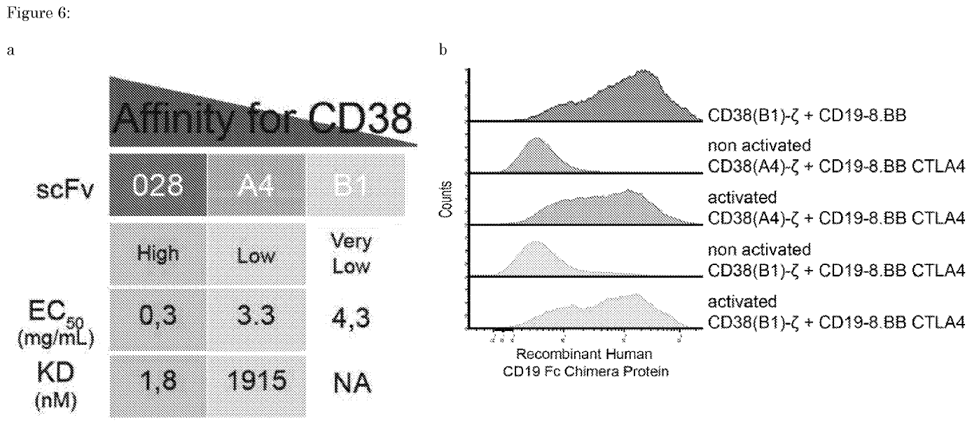

Interestingly, in long-term proliferation assays, the BCMA-8.28ζ+CD38-8.BB CTLA4 T cells showed an enhanced proliferative capacity, as compared to the BCMA-8.28ζ+CD38-8.BB T cells, that was not mediated through the costimulatory signals as both constructs share the same domains for co-stimulation (FIG. 5a). The enhanced proliferative capacity of the double-targeting BCMA-CAR+CD38−CAVI-R T cells outperformed the respective single targeting CARs (BCMA-8.28ζ), as well as the BCMA-28.28ζ and BCMA-8.BB3 CAR T cells (FIG. 5a). The enhanced proliferative capacity of the BCMA-CAR+CD38−CAVI-R T cells was corroborated with lower surface expression of programmed cell death protein 1 (PD-1), compared to BCMA-8.28ζ CAR T cells (FIG. 5b). The highly proliferating BCMA-CAR+CD38−CAVI-R T cells were characterized by the retention of a central memory phenotype, as a relative lower percentage of BCMA-CAR+CD38-CAVI-R T cells differentiated to a CD45RA⁻CD62L⁻ effector memory phenotype, as compared to the BCMA-CAR+CD38−CCR T cell population (FIG. 5c, d).

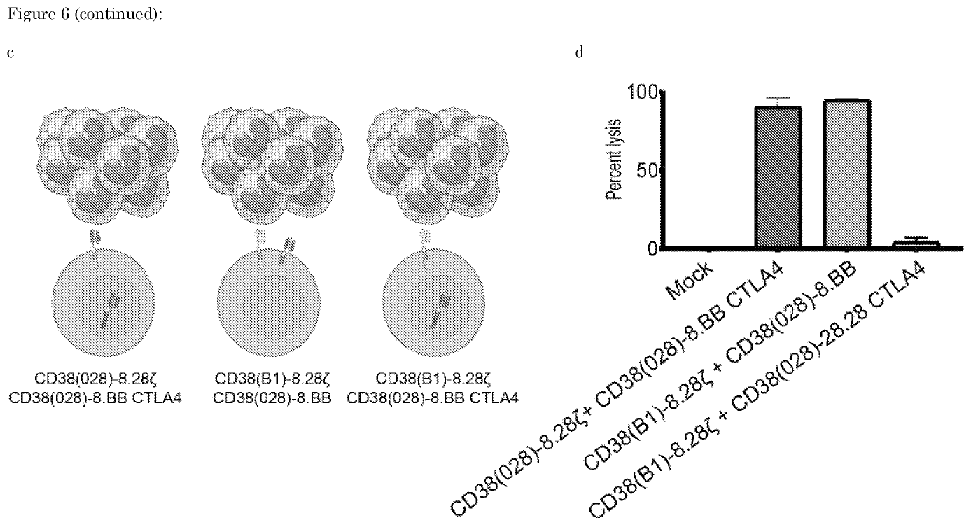

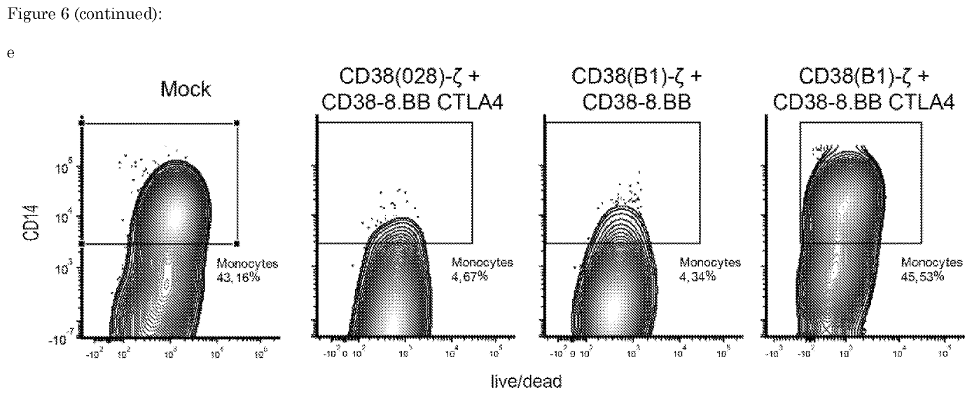

Next, the ability to achieve selective expression of the CAVI-R only after specific recognition of tumor cells expressing the CAR target, was determined. To this end, two single-chain variable fragments (scFvs) binding to the same CD38 epitope with gradually lower affinity were used (FIG. 6a) and a CD19 scFv was cloned into the CAVI-R construct. CD38−CAR+CD19−CAVI-R T cells were co-cultured with a tumor cell line expressing high level of CD38. We found that even very low affinity (scFv B1, dissociation constant (KD)=NA) binding of the CAR to highly expressed CD38 results in induction of the CAVI-R construct (FIG. 6b).

It was next evaluated whether lowering the binding affinity of the CAR could ensure selectivity of tumor-targeting, leaving intact healthy cells expressing low levels of CD38. Monocytes express CD38 at low levels, and we have previously shown that they are not lysed by a CD38(B1)−CAR. We evaluated the potential targeting and subsequent lysis of the monocyte subset from the low affinity CD38 CAR+high affinity CD38 CAVI-R T cells. Primary CD14+ monocytes were used as target cells and these were co-cultured them for 48 hours together with double-transduced T cells with high affinity CD38(028)−CAR+CD38−CAVI-R [CD38 (028)-8.28ζ+CD38-8.BB CTLA4], low affinity CD38(B1)−CAR+CD38−CCR [CD38(B1)-8.28ζ+CD38-8.BB] and low affinity CD38(B1)−CAR+CD38−CAVI-R [CD38(B1)-8.28ζ+CD38-8.BB CTLA4] (FIG. 6c). It was observed that binding of the low affinity CD38 (B1) to low expressed CD38 on the monocyte surface was not sufficient to induce CAVI-R expression and thus to induce cytotoxicity (FIG. 6d, e). On the contrary, T cells transduced to express either the high affinity CAR (CD38(028)-8.28ζ+CD38-8.BB CTLA4) or the permanently express CCR (CD38 (028)-8.28ζ+CD38-8.BB) eliminated the CD14 positive monocyte population (FIG. 6d, e).

1. An inducible chimeric co-stimulatory receptor (CCR) comprising:
an antigen specific binding domain;
a transmembrane domain;
one or more intracellular co-stimulatory signaling domains; and
an intracellular CTLA4 domain as an intracellular T cell activation dependent domain.

2. The inducible CCR according to claim 1, wherein the T cell activation dependent domain comprises a phosphorylation site.

3. The inducible CCR according to claim 1, wherein the intracellular co-stimulatory domain is an intracellular 4-1BB and/or an intracellular CD28 domain, and wherein the transmembrane domain is a CD28 or CD8α transmembrane domain.

4. The inducible CCR according to claim 1, wherein the antigen specific binding domain binds an antigen selected from the group consisting of the antigens listed in Table 1.

5. An immune cell expressing the inducible CCR according to claim 1.

6. The immune cell according to claim 5 further comprising a chimeric antigen receptor (CAR), wherein the CAR comprises:
an antigen specific binding domain;
a transmembrane domain;
optionally one or more intracellular co-stimulatory signaling domains; and
an intracellular primary signaling domain.

7. The immune cell according to claim 6, wherein the antigen specific binding domain of the CAR binds a first antigen and the antigen specific binding domain of the inducible CCR binds a second antigen, wherein the first antigen differs from the second antigen, or wherein the first and second antigen are the same but the binding affinities of the CAR and the inducible CCR to said first and second antigen differ.

8. The immune cell according to claim 6, wherein:
the antigen specific binding domain of the CAR is a single chain binding domain,
the antigen specific binding domain of the inducible CCR is a single chain binding domain, or
both the antigen specific binding domain of the CAR is a single chain binding domain and the antigen specific binding domain of the inducible CCR is a single chain binding domain.

9. The immune cell according to claim 6 wherein the CAR and inducible CCR both have a transmembrane CD8α domain.

10. A nucleic acid molecule that enables expression of an inducible chimeric co-stimulatory receptor (CCR) as defined in claim 1 in a suitable immune cell.

11. The nucleic acid molecule of claim 10, which is present in a vector.

12. A pharmaceutical composition, comprising the immune cell according to claim 5.

13. A method of producing an immune cell comprising:
providing immune cells, and
modifying the immune cells by enabling expression of a CAR and an inducible CCR as defined in claim 1.

14. A method of treating a malignancy in a patient, the method comprising
providing immune cells;
modifying the immune cells by enabling expression of a CAR and an inducible CCR as defined in claim 1; and
administering the modified immune cells to the patient.

15. A method of treating a malignancy in a patient, the method comprising the administration of the pharmaceutical composition of claim 12, optionally in combination with surgery, radiation therapy, one or more further anti-cancer drugs or a combination thereof.

16. The inducible CCR according to claim 1, wherein the intracellular CTLA4 domain comprises at least a YVKM motif.

17. The inducible CCR according to claim 16, wherein intracellular CTLA4 domain comprises the sequence TTGVYVKMPPT.

18. The immune cell according to claim 7, wherein the first and second antigen are selected from the group consisting of the antigens listed in Table 1.

19. The method according to claim 14, wherein the malignancy is a hematological malignancy or a solid tumor.

20. The method according to claim 14, wherein the immune cells are T cells or NK cells.
